(19) United States
(12) Patent Application Publication
(10) Pub. No.: US 2023/0395236 A1
(43) Pub. Date: Dec. 7, 2023

(54) System, Devices, and Methods for Generating Plant Ecosystems, Interactive Plant Avatars, and Virtual Social Communities in a Network Environment

(71) Applicants: Greyson S. Diaz, Seattle, WA (US); Eugene Washington, III, Auburn, WA (US); Angela Z. Troy, Renton, WA (US)

(72) Inventors: Greyson S. Diaz, Seattle, WA (US); Eugene Washington, III, Auburn, WA (US); Angela Z. Troy, Renton, WA (US)

(21) Appl. No.: 18/205,552

(22) Filed: Jun. 4, 2023

Related U.S. Application Data
(60) Provisional application No. 63/349,118, filed on Jun. 5, 2022.

(51) Int. Cl. G16H 20/70; G06T 13/40; G06F 3/04815

(52) U.S. Cl. CPC G16H 20/70; G06T 13/40; G06F 3/04815

(57) ABSTRACT

Systems, devices, and methods are described for providing, among other things, a plant community component including electrical circuitry configured to generate one or more garden attributes and one or more garden community recommendations based on at least one input indicative of a target garden characteristic and at least one input indicative of a calmness state. Also described are systems, devices, and methods that enable diverse users to actively engage in environmental stewardship and connect to tight-knit communities with a common interest: plants, healing the planet, and dignifying the human condition.

### RELATED APPLICATIONS

This application claims the benefit of priority under 35 USC § 119(e) of U.S. Provisional Patent Application No. 63/349,118 filed Jun. 5, 2022, the contents of which are incorporated herein by reference in their entirety.

### SUMMARY

In an aspect, the present disclosure is directed to, among other things, a system including a plant community component including electrical circuitry configured to generate one or more garden attributes and one or more garden community recommendations based on at least one input indicative of a target garden characteristic and at least one input indicative of a calmness state. In an embodiment, the system includes a virtual community component including electrical circuitry configured to generate a virtual community environment on one or more client devices and to enable communication and digital content exchanges between multiple users in different physical locations.

In an aspect, the present disclosure is directed to, among other things, a wellness system including a plant assessment component including electrical circuitry configured to evaluate one or more digital images of a plant and to determine a plant wellness potential profile based on a plant potential classifier trained to evaluate indicators of plant morphology and to predict a plant potential to improve health, elicit calmness, induce relaxation, increase alertness, mitigate fatigue, mitigate stress, or mitigate anxiety. In an embodiment, the wellness system includes a garden architecture component including electrical circuitry configured to generate one or more garden design recommendations based on at least one determined plant wellness potential.

In an aspect, the present disclosure is directed to, among other things, a system for generating a digital plant. In an embodiment, the system for generating the digital plant includes a plant image prediction unit including electrical circuitry configured to extract latent vector information from a plurality of images, combine the latent vector information, and generate a digital plant image which incorporates the combined latent vector information, and which has one or more characteristics of each of the plurality of images. In an embodiment, at least one of the plurality of images comprises a digital image of a human face. In an embodiment, the plant image prediction unit comprises electrical circuitry having at least one processor configured to generate a digital plant image which incorporates a characteristic from the image of a human face and an image of a plant.

In an aspect, the present disclosure is directed to, among other things, an interactive plant avatar system. In an embodiment, the interactive plant avatar system includes a plant personality unit including electrical circuitry configured to generate digital content consistent with a personality attribute set. In an embodiment, the interactive plant avatar system includes a plant avatar unit including electrical circuitry configured to generate an interactive plant avatar on one or more client devices, the interactive plant avatar having one or more physical attributes inferred from the personality attribute set. In an embodiment, the plant personality unit includes electrical circuitry configured to generate digital content consistent with a physical attribute set; and the interactive plant avatar unit includes electrical circuitry configured to generate an interactive plant avatar on one or more client devices, the interactive plant avatar having one or more personality attributes inferred from the personality attribute set and one or more physical attributes inferred from the physical attribute set.

In an aspect, the present disclosure is directed to, among other things, a metaverse system. In an embodiment the metaverse system includes a metaverse server coupled to a client computer, and a garden metaverse configuration component configured to generate a metaverse including one or more virtual plants having a geometric shape based on one or more inputs indicative of a wellness attribute. In an embodiment, the metaverse is accessible by avatars of users that access the metaverse via client devices.

In an aspect, the present disclosure is directed to, among other things, a wellness metaverse system. In an embodiment, the wellness metaverse system includes electrical circuitry including a hardware processor, a display, and a graphical user interface (GUI) configured to render and access a wellness metaverse. In an embodiment, the GUI includes a menu of selectable room attributes rendered on the display of a room within the wellness metaverse. In an embodiment, a user operates the GUI to navigate to a virtual garden metaverse space.

In an aspect, the present disclosure is directed to, among other things, a metaverse client terminal for providing a metaverse virtual space capable of enabling interaction between users in a plant community.

### BRIEF DESCRIPTION OF THE FIGURES

FIG. 1A is a schematic diagram of a system according to one embodiment.

FIG. 1B is a schematic diagram of a mobile app, onboarding, user-management workflow according to one embodiment.

FIG. 1C is a schematic diagram of a mobile app, onboarding, user-management component according to one embodiment.

FIG. 1D is a schematic diagram of a mobile app, onboarding, user-management component according to one embodiment.

FIG. 1E is a schematic diagram of a mobile app, onboarding, user-management component according to one embodiment.

FIG. 1F is a schematic diagram of a mobile app, onboarding, user-management component according to one embodiment.

FIG. 1G is a schematic diagram of a mobile app, onboarding, user-management component according to one embodiment.

FIG. 1H is a schematic diagram of a mobile app, onboarding, user-management component according to one embodiment.

FIG. 1I is a schematic diagram of a mobile app, onboarding, user-management component according to one embodiment.

FIG. 2 is a schematic diagram of a wellness system according to one embodiment.

FIG. 3 is a schematic diagram of a system for generating a digital plant ecosystem according to one embodiment.

FIG. 4 shows a flow diagram of a method according to one embodiment.

FIG. 5 is a schematic diagram of an interactive plant avatar system according to one embodiment.

FIG. 6 is a schematic diagram of a system according to one embodiment.

FIG. 7A is a schematic diagram of a system for generating a digital plant ecosystem according to one embodiment.

FIG. 7B is a schematic diagram of a user experience layout for a plant community experience according to one embodiment.

FIG. 7C is a schematic diagram of a mobile app layout for a plant community experience according to one embodiment.

FIG. 7D is a schematic diagram of a mobile app workflow according to one embodiment.

FIG. 8 is a schematic diagram of a system according to one embodiment.

FIG. 9A is a schematic diagram of a metaverse system according to one embodiment.

FIG. 9B is a Leaf Shape Map reproduced from Genki-Kûkan™ research initiative (part 1) reproducing slices of nature in the laboratory: Toyota global site: Frontier research. Toyota Motor Corporation Global Website. (n.d.). Retrieved May 9, 2022, from https://www.toyota-global.com/innovation/partner_robotinews/20201222_01.html.

FIG. 10 is a schematic diagram of a wellness metaverse system according to one embodiment.

FIG. 11 is a schematic diagram of a metaverse client terminal according to one embodiment.

FIG. 12A is a schematic diagram of a plant IOT system according to one embodiment.

FIG. 12B is a schematic diagram of a mobile app, onboarding, user-management component according to one embodiment.

FIG. 12C is a schematic diagram of a mobile app, onboarding, user-management component according to one embodiment.

FIG. 12D shows a diagram of a mobile app according to one embodiment.

FIG. 12E is a schematic diagram of a user experience layout according to one embodiment.

FIG. 12F is a schematic diagram of a mobile app workflow according to one embodiment.

FIG. 12G is a schematic diagram of a mobile app workflow according to one embodiment.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented here.

### DETAILED DESCRIPTION

The physical and social isolation measures implemented to reduce the spread of COVID-19 have had the unintended consequence of socially isolating millions. The sustained “social distancing” and “sheltering in place” has resulted in worsening what is often referred to as the loneliness epidemic or loneliness pandemic. For example, 61% of young adults aged 18 to 25 reported elevated levels of loneliness. Weissbourd R. et al., Loneliness in America how the Pandemic Has Deepened an Epidemic of Loneliness and What We Can Do about it? [(accessed on 14 May 2022)]; Available online: https://mcc.gse.harvard.edu/reports/loneliness-in-America. Over a quarter of people over age 60 live alone in the United States, and more than 43 percent of them reported feeling lonely even before the COVID-19 pandemic. See e.g., Dangerfield-Cha, M., & Zhang, J. (2021). Solving the Loneliness Epidemic, Two Generations at a Time. Stanford Social Innovation Review. https://doi.org/10.48558/Q5N3-0503. The physical and social isolation measures may have also resulted in the growth of hyper-individualism and the need to re-socialize and promote technologies for forming communities.

Accordingly, in an embodiment, the disclosed technologies and methodologies include components, hardware, firmware, software, drivers, utilities, mobile applications, and the like working in concert to enable diverse users to actively engage in healing the planet and dignifying the human condition. In an embodiment, the disclosed technologies and methodologies include systems, devices, and methods directed to improve mental health and promote environmental stewardship by providing hardware, firmware, software that enables a tight-knit community of users with a common interest such as plants and gardening to engage in a virtual environment. For example, in an embodiment, the disclosed technologies and methodologies include systems, devices, and methods for improving mental health by enabling meaningful community via exchange platforms supporting the cultivation of customized indoor garden spaces.

In an embodiment, the disclosed technologies and methodologies include components, hardware, firmware, software, drivers, utilities, and the like working in concert to enable diverse users to actively engage in environmental stewardship and connect to tight-knit communities with a common interest: plants, healing the planet, and dignifying the human condition. In an embodiment, the disclosed methodologies or technologies are directed to user-specific and enterprise-wide planet stewardship systems, devices, and methods.

In an embodiment, the disclosed technologies and methodologies include systems, devices, and methods configured to allow users to engage in a way that creates community, meaningful relationships, while promoting environment stewardship. For example, users can use the disclosed technology platforms to develop their interest in gardening and environmental sustainability and form a tight knit community under common interests and specialized roles.

In an embodiment, the disclosed methodologies or technologies are configured to leverage mental health technology, machine learning, and diagnostic protocols to engage users in activities that strengthen and personalize the relationship between a caretaker and their plant, and that connects members to a tightknit community that shares common sustainability, health, and wellness interests.

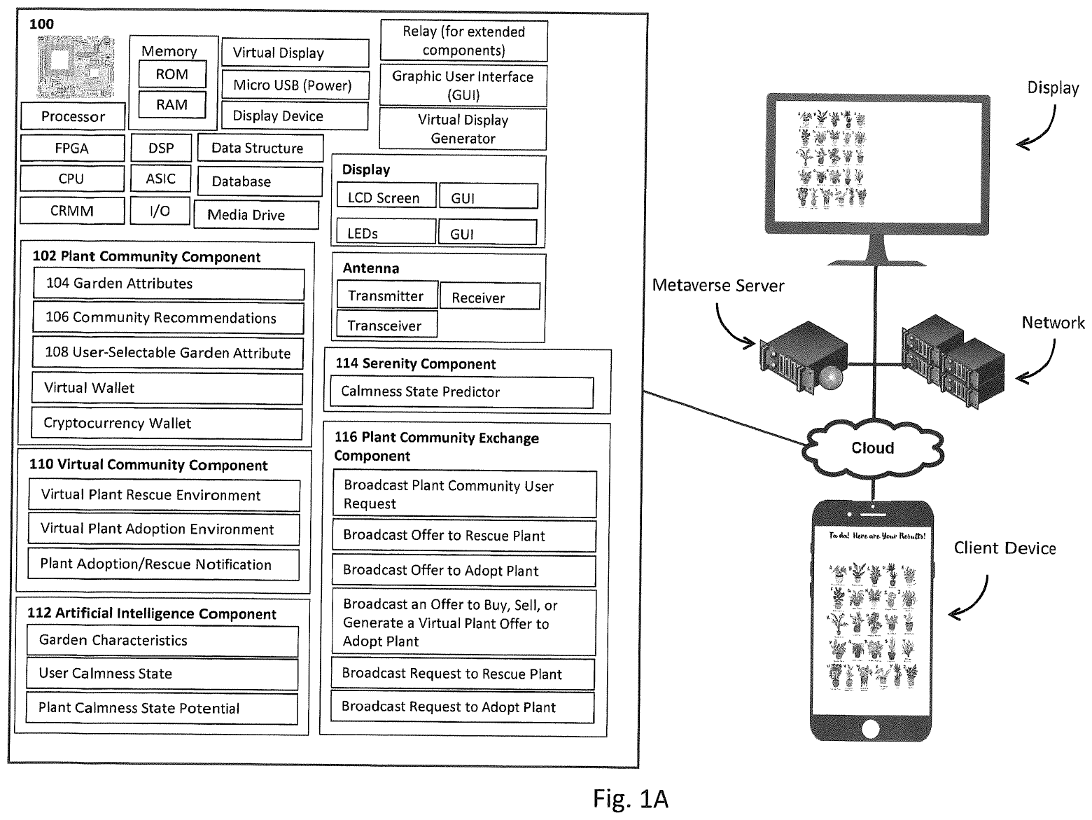

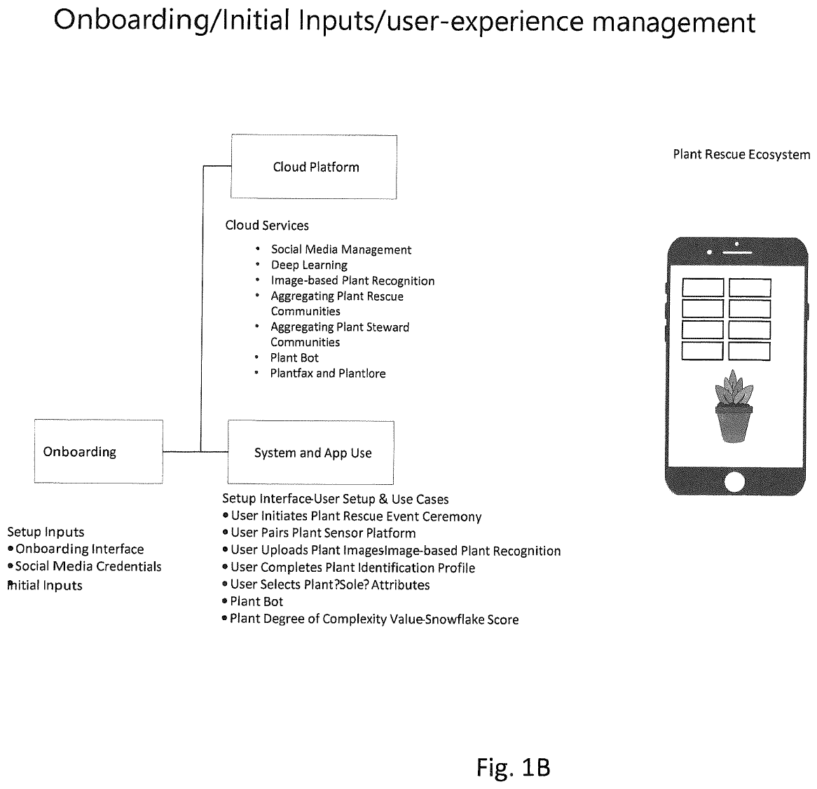

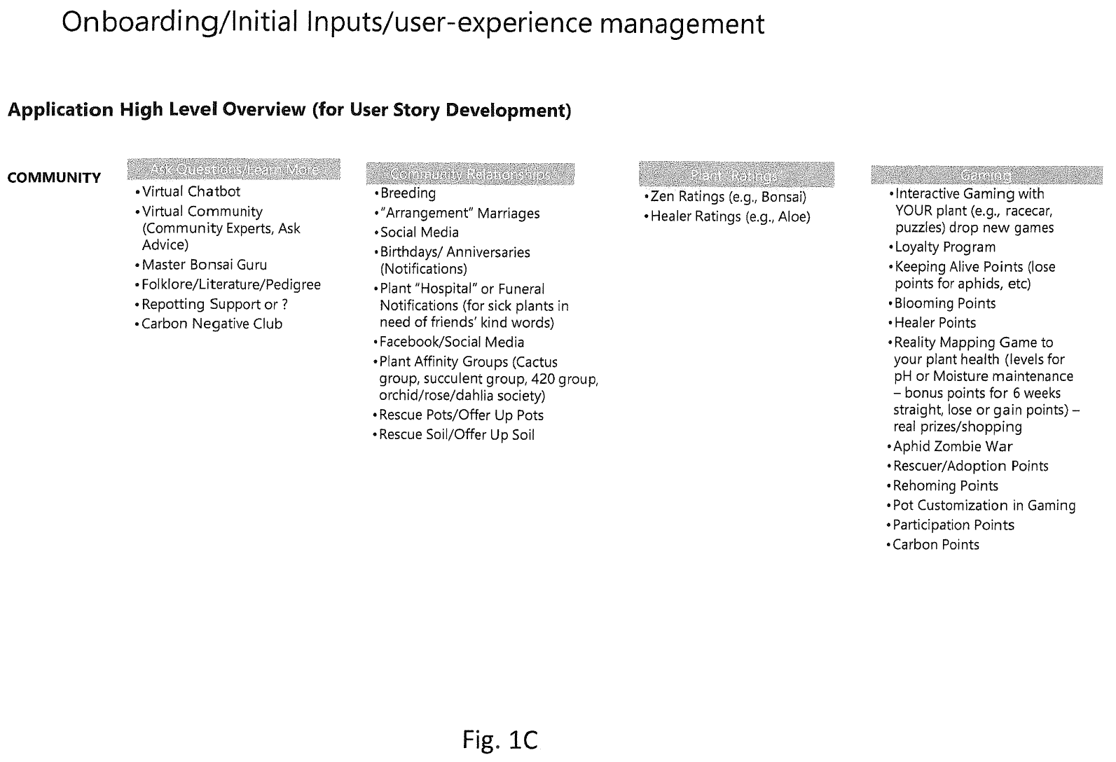

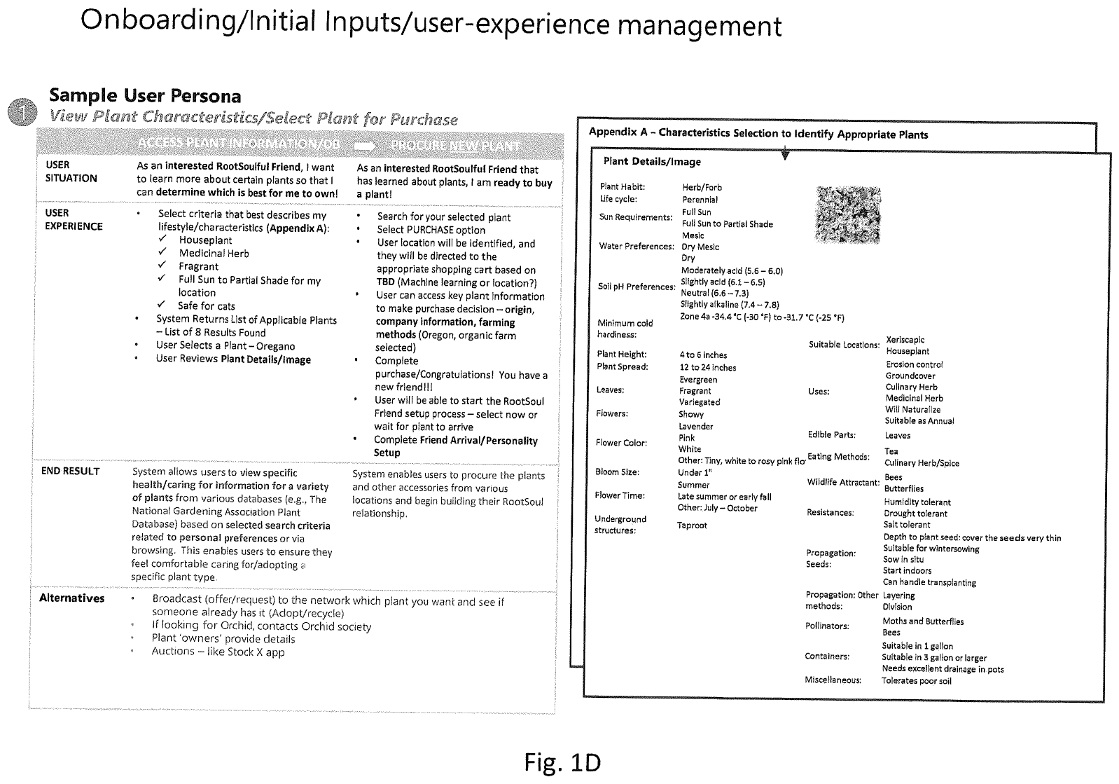

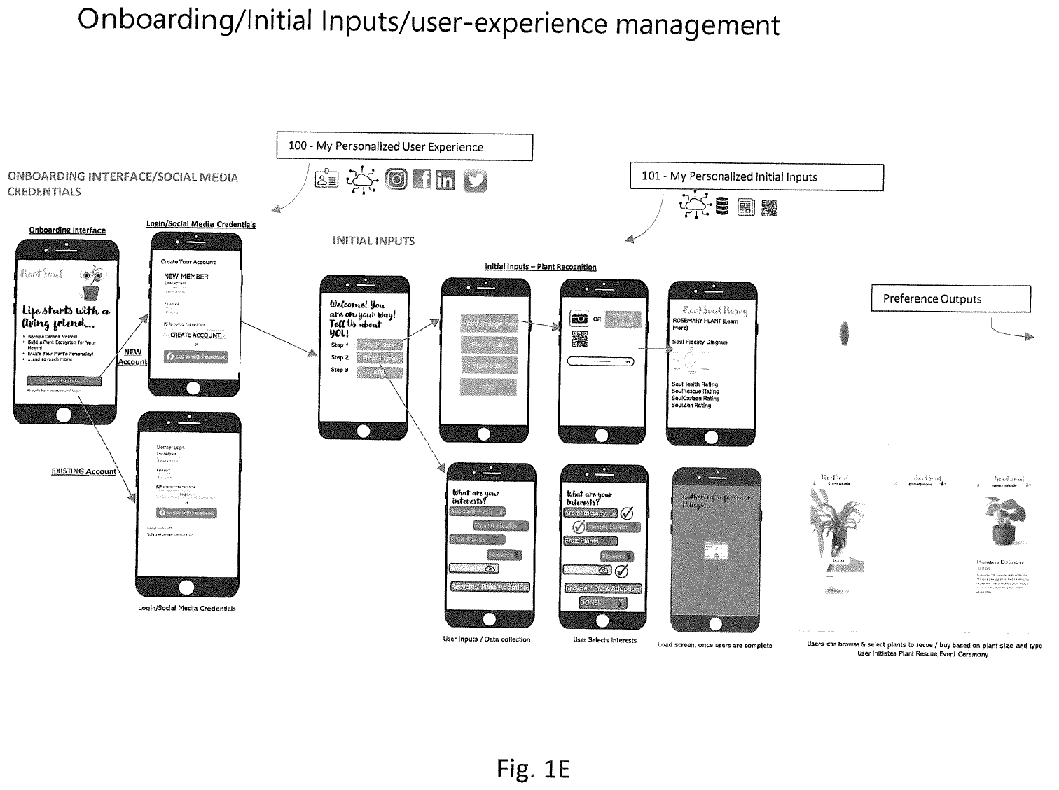

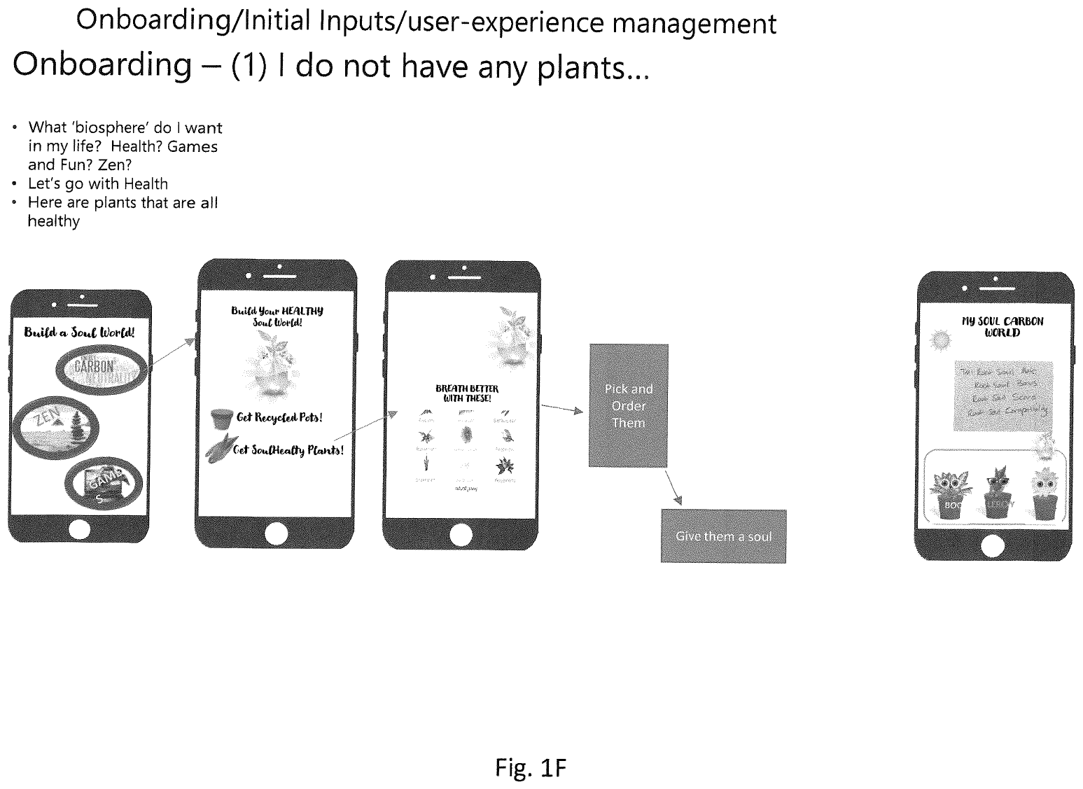

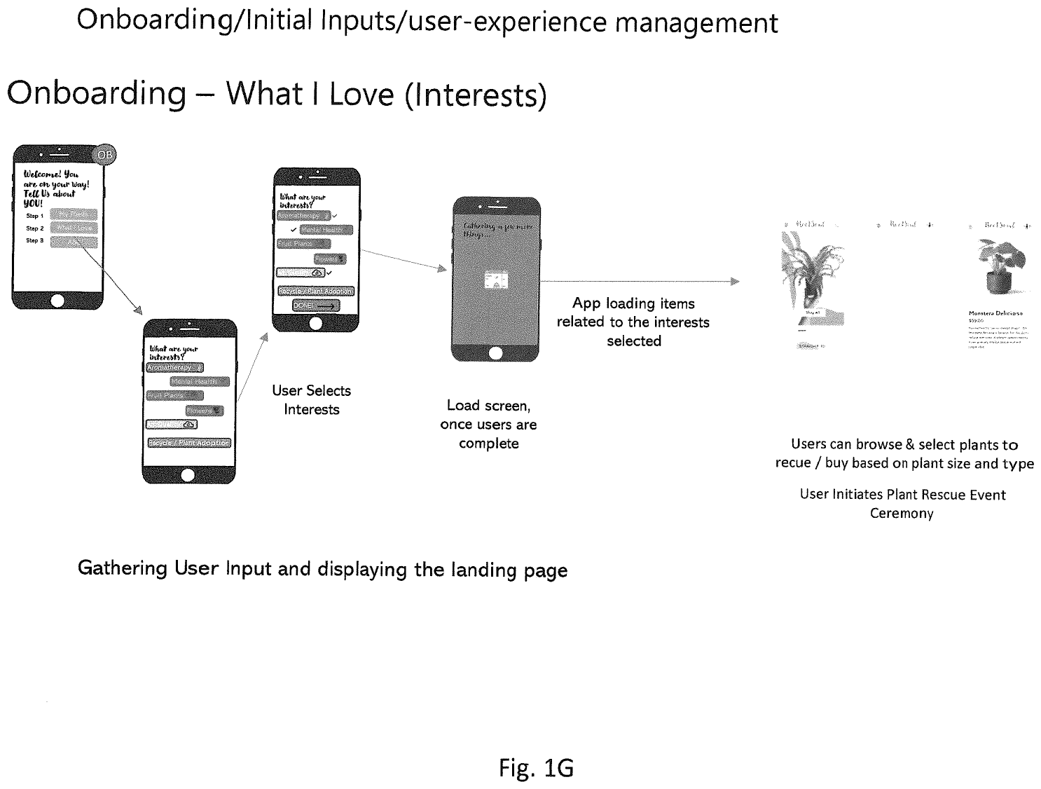

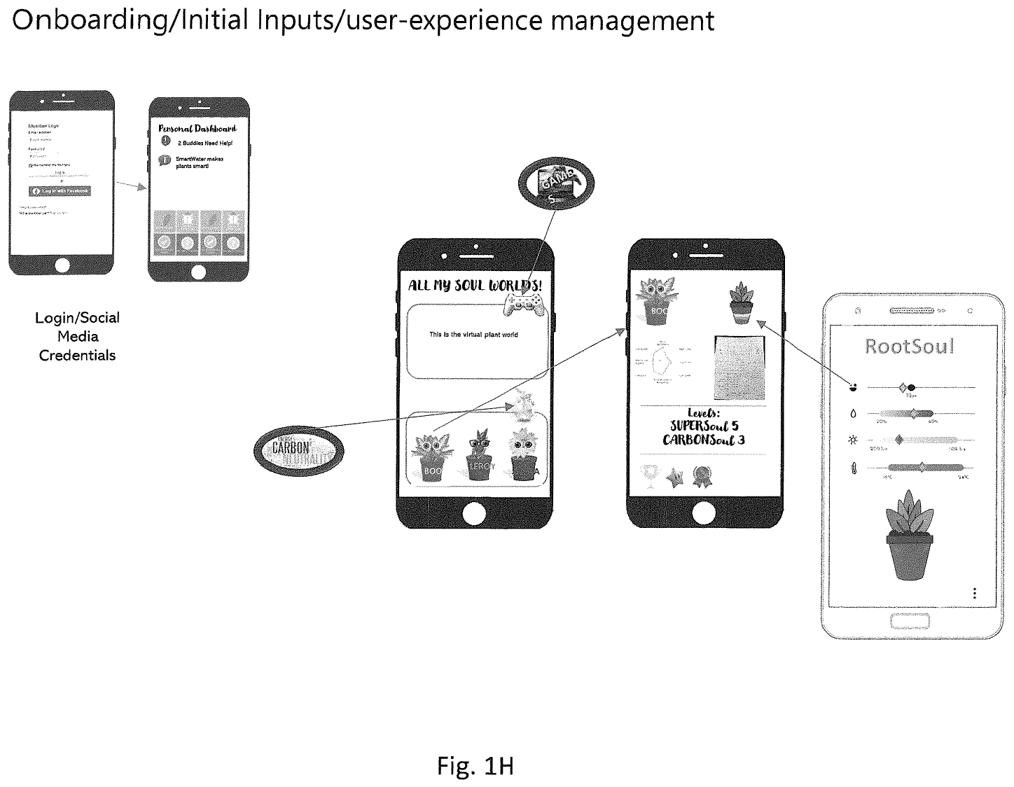

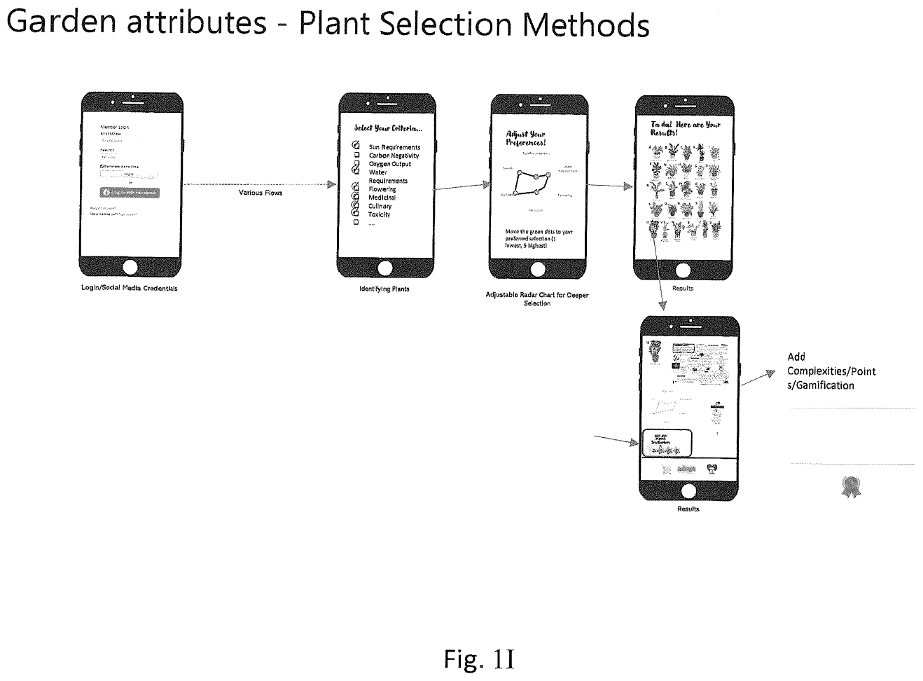

FIGS. 1A-1I show a system 100 in which one or more of the disclosed methodologies or technologies can be implemented such as, for example, generating a digital plant ecosystem, generating a plant community ecosystem, providing metaverse spaces capable of enabling interaction between users in a plant community, rendering interactive plant avatars, enabling wellness metaverse systems, and the like. In an embodiment, the system 100 includes a plant community component 102 having electrical circuitry 104 configured to generate (determine, assess, calculate, predict, derive, or the like) one or more instances of a garden attribute on a virtual display, a user interface, a command line user interface, a menu driven user interface, a graphical user interface, a display, a user input device, a system of interactive visual components, or the like. In an embodiment, the system 100 includes a plant community component 102 having electrical circuitry 106 configured to generate one or more instance of a garden community recommendation on a virtual display, a user interface, a command line user interface, a menu driven user interface, a graphical user interface, a display, a touch-sensitive display, a user input device, a system of interactive visual components, or the like. For example, in an embodiment, the plant community component 102 includes electrical circuitry 108 configured to generate a user-selectable garden attribute or a user-selectable garden community recommendation, on a display of a client device, based on at least one input via an input component (e.g., a selectable menu on a display, an audio input device, a gesture input device, an Alexa enable device, a user interface, or the like) indicative of a target garden characteristic and at least one input indicative of a calmness state. In an embodiment, the plant community component 102 includes electrical circuitry 108 configured to generate visual outputs such as graphics, icons, text, user-interactive graphical user interface objects, videos, virtual menus, and the like.

In an embodiment, system 100 includes electrical circuitry configured to provide an input interface and an output interface between the device and a user. See e.g., U.S. Pat. No. 11,327,648 (which content is incorporated herein by reference in full.) For example, in an embodiment, system 100 includes a display controller configured to receive or send electrical signals to and from the touch-sensitive display. In an embodiment, the includes electrical circuitry configured to display visual output to the user. Non-limiting examples of visual output include digital content, graphics, icons, text, user-interactive graphical user interface objects, videos, virtual menus, and the like

In an embodiment, system 100 includes a graphical user interface object configured to respond to inputs directed toward the graphical user interface object. Non-limiting examples of graphical user interface objects include, among other things, buttons, hyperlinks, icons, interface controls, selectable menu items, sliders, switches, and the like. See e.g., U.S. Pat. No. 11,327,648 (which content is incorporated herein by reference in full). See e.g., U.S. Pat. No. 11,327,648 (which content is incorporated herein by reference in full).

In an embodiment, system 100 includes electrical circuitry including a hardware processor, a display, and a user interface configured to render and access a plant community ecosystem, a wellness metaverse, a virtual garden system and the like. Non-limiting examples of a user interface include a virtual display, an interactive virtual display, a user interface, a command line user interface, a menu driven user interface, a graphical user interface, a display, a user input device, a system of interactive visual components, and the like. For example, in an embodiment, the system 100 includes electrical circuitry including a hardware processor, a display, and a user interface configured to render and access a menu of selectable room attributes rendered on the display of a room within the wellness metaverse. In an embodiment, a user operates the graphical user interface (GUI) to navigate to a garden metaverse space.

In an embodiment, system 100 includes electrical circuitry configured to render and access a plant community ecosystem, a wellness metaverse, a virtual garden system and the like. For example, in an embodiment, system 100 includes electrical circuitry configured to initiate a discovery protocol that allows a client device and the system 100 to identify each other and negotiate one or more pre-shared keys. Non-limiting examples of client devices include application interface with smart devices, cellular phones, client devices configured to function in a virtualized environment, computer devices, desktop computer devices, IOT devices, laptop computer devices, managed node devices, mobile thin client devices, notebook computer devices, PDA devices, personal computers, remote controllers, smart devices, smart eyewear devices, smart phones, smart wearable devices, tablet devices, Virtual Reality (VR) headsets, VR hardware, wearable devices, workstations, and the like.

In an embodiment, the plant community component 102 includes an artificial intelligence component 112 trained to detect indicators of a user calmness state and to determine garden characteristic based on a plant morphology-plant wellness potential correlation policy, a plant calmness state potential correlation policy, or the like. In an embodiment, the artificial intelligence component 112 is trained to predict a user calmness state and to determine a garden characteristic including a leaf size percent distribution, leaf shape percent distribution, or leaf shape color percent distribution, or combinations thereof, based on the user's calmness state.

In an embodiment, the plant community component 102 includes electrical circuitry including a hardware processor, a display, and a user interface configured to render and access a plant community ecosystem, a wellness metaverse, a virtual garden system and the like on a display. For example, in an embodiment, the plant community component 102 includes electrical circuitry including a display for rendering, presenting, accessing, and the like interactive digital content. Non-limiting examples of displays include augmented reality displays, head-mounted displays, retinal projectors, retinal displays, retinal scan displays, virtual reality displays, virtual reality headsets, virtual retinal displays, and the like. In an embodiment, the plant community component 102 includes a plant community ecosystem component configured to render and access to a plant community ecosystem, a wellness metaverse, a virtual garden system and the like on a display.

In an embodiment, system 100 includes a retinal display configured to project digital content directly onto the retina of the eye. For example, in an embodiment, the retinal display includes electrical circuitry, light-emitting diodes, optical waveguides, and the like configured to generate, steer, manipulate, etc. photons to render a panoramic, high resolution, color digital content, and to project the digital content directly onto the retina of the eye. In an embodiment, system 100 is operably coupled to a display configured to project digital content directly onto the retina of the eye. For example, in an embodiment, a retinal display includes computational circuitry, light-emitting diodes, optical waveguides, and the like configured to generate, steer, manipulate, etc. photons to render a panoramic, high resolution, color digital content, and to project the digital content directly onto the retina of the eye.

In an embodiment, the plant community component 102 includes electrical circuitry configured to generate one or more garden attributes and one or more garden community recommendations based on at least one input indicative of a calmness state.

Target Garden Characteristic

In an embodiment, the plant community component 102 includes electrical circuitry configured to generate at least one of a garden attribute, or a garden community recommendation, based on at least one input indicative of a target garden characteristic and at least one input indicative of a calmness state.

For example, in an embodiment, the plant community component 102 includes electrical circuitry configured to generate a one or more virtual selection menus on a user interface that allows a user to select a target garden characteristic and at least one input indicative of a calmness state. In an embodiment, the plant community component 102 includes electrical circuitry configured to generate at least one of a garden attribute or a garden community recommendation, and to generate a one or more virtual selection menus on a user interface that allows a user to select at least one of a garden attribute or a garden community recommendation, based on at least one input indicative of a target garden characteristic and at least one input indicative of a calmness state.

Non-limiting examples of target garden characteristics include garden health information, health objectives information, exposome objectives information, and the like, or combinations thereof. Further non-limiting examples of target garden characteristics include pollution abatement information, indoor environment information, indoor air purification information, and the like, or combinations thereof. Further non-limiting examples of target garden characteristics include garden geolocation information, garden care level information, garden exposome information, and the like, or combinations thereof. Further non-limiting examples of target garden characteristics include plant morphology information including leaf size information, leaf shape information, leaf color information, leaf size percent distribution information, leaf shape percent distribution information, or leaf shape color percent distribution information, and the like, or combinations thereof. In an embodiment, the system 100 includes a plant community component 102 including electrical circuitry configured to generate one or more garden attributes and one or more garden community recommendations based on geolocation information and at least one input indicative of a target garden characteristic and at least one input indicative of a calmness state.

Non-limiting examples of geolocation information include geocoding information, geographic location information, geolocation information, geo-positioning information, geotagging information, georeferencing information, and the like.

In an embodiment, the target garden characteristic includes a threshold characteristic, a presence or absence of a characteristic, a not to exceed characteristic, a maximum level characteristic, and the like. For example, in an embodiment, the target garden characteristic includes target garden health characteristic. In an embodiment, the target garden characteristic includes target health objectives characteristic. In an embodiment, the target garden characteristic includes target pollution abatement characteristic, a target indoor environment characteristic, a target indoor air purification characteristic. In an embodiment, the target garden characteristic includes one or more inputs associated with target garden health information. In an embodiment, the target garden characteristic includes one or more inputs associated with target health objectives. In an embodiment, the target garden characteristic includes one or more inputs associated with target pollution abatement information, target indoor environment information, target indoor air purification information, or the like.

In an embodiment, the target garden characteristic includes one or more inputs associated with geolocation information. In an embodiment, the target garden characteristic includes one or more inputs associated with exposome information. In an embodiment, the target garden characteristic includes one or more inputs associated with target garden care level information. In an embodiment, the target garden characteristic includes target plant morphology information. In an embodiment, the target garden characteristic includes a target leaf size, a target leaf shape, a target leaf color. In an embodiment, the target garden characteristic includes target leaf size percent distribution information, target leaf shape percent distribution information, or target leaf shape color percent distribution information, or combinations thereof.

Calmness State

In an embodiment, the plant community component 102 includes electrical circuitry configured to determine at least one of a garden attribute, or a garden community recommendation, based on at least one input indicative of a calmness state of a user. Non-limiting examples of an input indicative of a calmness state include inputs indicative of an anxiety state, a stress state, an alertness state, an anxiety level, a stress level, an agitation level, or an excitement level, a metal health state, and the like. In an embodiment, the plant community component 102 is configured to generate one or more garden attributes and the one or more garden community recommendations responsive to the least one input indicative of the target garden characteristic and at least one biometric input indicative of an anxiety state, a stress state, or an alertness state. In an embodiment, the plant community component 102 is configured to generate one or more garden attributes and the one or more garden community recommendations responsive to the least one input indicative of the target garden characteristic and at least one input indicative of an anxiety level, a stress level, an agitation level, or an excitement level. In an embodiment, the plant community component 102 includes electrical circuitry configured to exchange and store target garden characteristic, anxiety level information, stress level, agitation level information, excitement level information, garden attribute information, garden community recommendations, or the like information from one or more client devices.

In an embodiment, the plant community component 102 is configured to generate one or more garden attributes and the one or more garden community recommendations responsive to at least one input indicative of an exposome condition. Non-limiting examples of exposome conditions include the presence, absence, levels, concentrations, sources, and the like of indoor or outdoor aeroallergens, air pollutants and the like. It has been noted that climate change, urbanization, loss of biodiversity, and the like “affect sources, emissions, and concentrations of main aeroallergens and air pollutants and are among the most critical challenges facing the health and quality of life of the still increasing number of allergic patients today and in the coming decades.” See e.g., Lorenzo Cecchi et al., J Allergy Clin Immunol. 2018 Mar.; 141(3):846-857. doi: 10.1016/j.jaci.2018.01.016. Retrieved Feb. 5, 2022, from https://pubmed.ncbi.nlm.nih.gov/29519451/. In an embodiment, the plant community component 102 is configured to generate one or more garden attributes and the one or more garden community recommendations responsive to at least one input indicative of an exposome condition and at least one input indicative of a calmness state.

In embodiment, an exposome condition includes a specific or nonspecific external environmental exposure conditions. In embodiment, an exposome condition includes a specific or nonspecific external environmental exposure condition and its consequences at the organ or cell levels. In embodiment, an exposome condition includes exosome conditions associated with an inflammatory response to allergens. In embodiment, an exposome condition includes environmental exposure conditions including changes in climate, the presence of air and water contaminates, pollutants, and the like. In embodiment, an exposome condition includes conditions indicative of environmental exposure to chemicals, metals, or particles that negatively or positively affect animal, plant, or human health. It has been noted that environmental exposures can alter the normal function of the endocrine system, can exert toxicity specifically on the nervous, hematopoietic, respiratory, or digestive systems, or can lead to adverse health effects by altering expression of noncoding RNA, including microRNA and long noncoding RNA. See e.g., https://doi.org/10.1016/B978-0-12-812433-8.00010-1.

In an embodiment system 100 includes a virtual community component 110 including electrical circuitry configured to generate a virtual community environment on one or more client devices and to enable communication and digital content exchanges between multiple users in different physical locations. In an embodiment, the virtual community component 110 includes electrical circuitry configured to enable multiple users in different physical locations to exchange virtual objects within a shared virtual garden environment.

In an embodiment system 100 includes a virtual community component 110 including electrical circuitry configured to initiate a discovery protocol that allows a client device and the virtual community component 110 to identify each other and negotiate one or more pre-shared keys.

In an embodiment, the virtual community component 110 includes electrical circuitry configured to enable multiple users in different physical locations to adopt virtual plants within a shared virtual plant rescue environment In an embodiment, the virtual community component 110 includes electrical circuitry configured to enable multiple users in different physical locations to be authenticated, authorized, and enabled to form smart contracts associated with an adopt virtual plants, within a shared virtual plant rescue environment. In an embodiment, the virtual community component 110 includes electrical circuitry configured to electronically notify at least one member of plant community an availability of a virtual or real plant for rescue.

In an embodiment, the virtual community component 110 includes electrical circuitry configured to send an electronic communication to at least one member of plant community indicative of an availability of a virtual or real plant for rescue. For Example, in an embodiment, the virtual community component 110 includes electrical circuitry configured to send a push notification to a device to provide a message regarding the availability of a virtual or real plant for rescue. In an embodiment, the virtual community component 110 includes electrical circuitry configured to send a short message services (SMS) message to a mobile phone of a user regarding the availability of a virtual or real plant for rescue. In an embodiment, the virtual community component 110 includes electrical circuitry configured to send an e-mail to an e-mail address of a user regarding the availability of a virtual or real plant for rescue. In an embodiment, the virtual community component 110 includes electrical circuitry configured to generate a virtual display that allows user to navigate through a catalogue of plants available for adoption, purchase, or rescue, and products that meet the user's needs specified through a menu of selectable choices.

In an embodiment, the virtual community component 110 includes electrical circuitry configured to facilitate payment to a digital wallet responsive to one or more inputs indicative of transaction for a plant adoption. In an embodiment, the virtual community component 110 includes electrical circuitry configured to initiate an increase in value to a cryptocurrency wallet responsive to one or more inputs indicative of an acceptance to an offer to sell a plant, rescue a plant, exchange a plant, exchange garden materials, or the like. In an embodiment, the virtual community component 110 includes electrical circuitry configured to initiate a cryptocurrency transfer to or from a cryptocurrency wallet based on a plant community user transaction. In an embodiment, the virtual community component 110 includes electrical circuitry configured to facilitate a virtual payment to a seller for an offer to sell a plant based on one or more inputs indicative of a selection of a checkout Uniform Resource Locator (URL) associated with a virtual selling or purchasing cart.

On-Boarding and Application Attributes

Referring to FIGS. 1B-1I, in an embodiment, system 100 includes an onboarding component including electrical circuitry configured to enable a mobile application (app) onboarding and user-experience management process. In an embodiment, the onboarding component includes a graphical user interface including user selectable icons representing onboarding setup inputs.

In an embodiment, system 100 includes a serenity component 114 including electrical circuitry configured to predict a calmness state. In an embodiment, the serenity component 114 includes a hardware processor, a display, and a user interface configured to predict a calmness state responsive to at least one input indicative of calmness state of a user. For example, in an embodiment, the serenity component 114 is configured to predict the calmness state based on at least one input indicative of an anxiety state, a stress state, an agitation level, or an excitement level. In an embodiment, the serenity component 114 is configured to predict the calmness state based on at least one biometric input. In an embodiment, the serenity component 114 is configured to predict the calmness state based on a menu selection input from a virtual display, a user interface, a command line user interface, a menu driven user interface, a graphical user interface, a display, a user input device, a system of interactive visual components, or the like. In an embodiment, the serenity component 114 is configured to predict the calmness state based on a sleep level indication. In an embodiment, the serenity component 114 is configured to predict the calmness state based on a rest level indication. In an embodiment, the serenity component 114 is configured to predict the calmness state based on user-specific cognitive information. In an embodiment, system 100 includes a serenity component 114 including electrical circuitry configured to predict a calmness state and a plant community component 102 including electrical circuitry configured to predict a garden configuration and to generate one or more garden community recommendations responsive to at least one input indicative of an environmental condition and at least one input indicative of a calmness state.

Garden Attributes In an embodiment, the plant community component 102 includes electrical circuitry configured to generate one or more garden attributes based on at least one input indicative of a target garden characteristic. Non-limiting examples of garden attributes include attributes indicative, descriptive, or the like of air filtering plants, pollution abatement plants, oxygenation plants, and the like. Further non-limiting examples of garden attributes include attributes indicative, descriptive, or the like of calming garden configurations, relaxation garden configurations, mediative garden configuration, and the like. In an embodiment, the plant community component 102 includes electrical circuitry configured to generate one or more garden attributes based on at least one input indicative of a target garden characteristic and at least one input indicative of the predicted calmness state.

In an embodiment, the plant community component 102 includes electrical circuitry configured to generate one or more garden attributes and one or more garden community recommendations based on at least one input indicative of a target garden characteristic and at least one input indicative of the predicted calmness state. In an embodiment, the plant community component 102 includes electrical circuitry configured to generate one or more garden attributes based on at least one input indicative of a target garden characteristic and at least one input indicative of a calmness state. In an embodiment, the plant community component 102 includes electrical circuitry including a hardware processor, a display, and a user interface configured to generate, render, access, and the like one or more garden attributes based on at least one input indicative of a target garden characteristic and at least one input indicative of a calmness state.

Referring to FIG. 1I, in an embodiment, the plant community component enables a mobile application that is configured to generate one or more garden attributes and one or more garden community recommendations and allow a user to electronically select one or more garden attributes such a plant to be rescued.

Referring to FIGS. 1B-1I, in an embodiment, the one or more garden attributes include at least one attribute indicative of an air filtering plant, a pollution abatement plant, an oxygenation plant. In an embodiment, the one or more garden attributes include a garden configuration predicted to abate pollution, a garden configuration predicted to filter air, or a garden configuration predicted to purify air in an indoor space. In an embodiment, the one or more garden attributes include a garden configuration predicted to oxygenate an indoor space. In an embodiment, the one or more garden attributes include an easy to care for garden configuration, or a minimal maintenance garden configuration. In an embodiment, the one or more garden attributes include a garden configuration predicted to promote health. In an embodiment, the one or more garden attributes include a garden configuration predicted to promote rest. In an embodiment, the one or more garden attributes include a garden configuration predicted to elicit calmness. In an embodiment, the one or more garden attributes include garden configuration predicted to improve concentration. In an embodiment, the one or more garden attributes include a fatigue reduction garden configuration.

In an embodiment, the one or more garden attributes include an insomnia prevention garden configuration. In an embodiment, the one or more garden attributes include a calming garden configuration, or a relaxation garden configuration. In an embodiment, the one or more garden attributes include a colorful garden configuration. In an embodiment, the one or more garden attributes include a garden configuration predicted to improve alertness. In an embodiment, the one or more garden attributes includes a target leaf shape distribution, a target leaf size distribution, a target leaf color. In an embodiment, the one or more garden attributes includes a target leaf shape percent. In an embodiment, the one or more garden attributes includes a target leaf size percent distribution, a target leaf shape percent distribution, or a target leaf shape color percent distribution, or combinations thereof.

Garden Community Recommendations

In an embodiment, the plant community component 102 includes electrical circuitry configured to determine one or more garden community recommendations based on at least one input indicative of a target garden characteristic, at least one input indicative of a calmness state, or the like. In an embodiment, the plant community component 102 includes electrical circuitry configured to generate one or more instances of a garden community recommendation on a virtual display, an interactive virtual display, a user interface, a command line user interface, a menu driven user interface, a graphical user interface, a display, a user input device, a system of interactive visual components, or the like. In an embodiment, the plant community component 102 includes electrical circuitry including a hardware processor, a display, and a user interface configured to generate, determine, render, predict, form, or the like a garden community recommendation on a virtual display, an interactive virtual display, a user interface, a command line user interface, a menu driven user interface, a graphical user interface, a display, a user input device, a system of interactive visual components, or the like.

In an embodiment, the one or more garden community recommendations include a one or more instance of garden community menu selection on a user interface, the menu selection including a plurality of user-selectable garden community recommendations, a plurality of user-selectable garden communities, a plurality of user-selectable garden community characteristics, or the like. In an embodiment, the one or more garden community recommendations include a one or more instance on a user interface to join a garden community. In an embodiment, the one or more garden community recommendations include an electronic request to join a garden community. In an embodiment, the one or more garden community recommendations include one or more instance of a virtual community recommendation. In an embodiment, the one or more garden community recommendations include an electronic communication request to join a virtual garden community. In an embodiment, the one or more garden community recommendations include a request to join a garden metaverse. In an embodiment, the one or more garden community recommendations include a request to join a garden multiverse community.

In an embodiment, the one or more garden community recommendations include access information (biometric factor data, cryptographic key information, key-based authentication data, passphrase information, password information, Personal Identification Number (PIN) information, security token information, smart card information, string of electronic characters, or the like) to engage with a virtual garden community, a garden metaverse, or a garden multiverse community. In an embodiment, the one or more garden community recommendations include one or more instances of an indoor-garden community recommendation. In an embodiment, the one or more garden community recommendations include one or more instances of a plant rescue community recommendation. In an embodiment, the one or more garden community recommendations include one or more instances of a plant cultivation utensils community recommendation. In an embodiment, the one or more garden community recommendations include one or more instances of a pollution abatement garden community, air-filtering garden community, an indoor air purification garden community, or an oxygenation garden community recommendation.

In an embodiment, the one or more garden community recommendations include one or more instances on a virtual display indicative of an easy to care for garden community, or a minimal maintenance garden community recommendation. In an embodiment, the one or more garden community recommendations include one or more instances of a garden that promotes health community recommendation. In an embodiment, the one or more garden community recommendations include one or more instances of a garden that promotes sleep community, a garden that promotes rest, a fatigue reduction garden community, an insomnia prevention garden community recommendation.

In an embodiment, the one or more garden community recommendations include one or more instances of a calming garden community, or a relaxation garden community recommendation. In an embodiment, the one or more garden community recommendations include one or more instances of a colorful garden community recommendation. In an embodiment, the one or more garden community recommendations include one or more instances of an improve concentration garden community recommendation. In an embodiment, the one or more garden community recommendations include one or more instances of an increase personal energy garden community recommendation.

In an embodiment, the plant community component 102 includes electrical circuitry configured to generate one or more instances of user-selectable garden community recommendations based on the at least one input indicative of the target garden characteristic and the at least one input indicative of a calmness state.

In an embodiment, the plant community component 102 includes electrical circuitry configured to provide an input interface and an output interface between the device and a user that allows the user to interface with the system and select from a set of garden community recommendations via one or more virtual menus or graphical user interface objects. For example, in an embodiment, the plant community component 102 includes a graphical user interface object configured to respond to inputs directed toward the graphical user interface that actuate a user-selected garden community recommendation.

In an embodiment, the plant community component 102 includes electrical circuitry configured to generate a user interface including the one or more instances of the user-selectable garden community recommendations based on the at least one input indicative of the target garden characteristic and the at least one input indicative of a calmness state. In an embodiment, the plant community component 102 includes electrical circuitry configured to generate a graphical display including the one or more instances of the user-selectable garden community recommendations based on the at least one input indicative of a target garden maintenance level and the at least one input indicative of a calmness state. In an embodiment, the plant community component 102 includes electrical circuitry configured to generate a graphical display including the one or more instances of the user-selectable garden community recommendations based on the at least one input indicative of an exposome characteristic.

In an embodiment, system 100 includes a broadcast component including electrical circuitry configured to broadcast a plant community user request. In an embodiment, system 100 includes a broadcast component including electrical circuitry configured to broadcast an offer to rescue a plant. In an embodiment, system 100 includes a broadcast component including electrical circuitry configured to broadcast an offer of a plant available for rescue. In an embodiment, system 100 includes a broadcast component including electrical circuitry configured to broadcast an offer to adopt a plant. In an embodiment, system 100 includes a broadcast component including electrical circuitry configured to communicate a notice placing a plant for adoption. In an embodiment, system 100 includes a broadcast component including electrical circuitry configured to broadcast an offer to sell a plant or to buy a plant. In an embodiment, system 100 includes a plant community exchange component 116 including electrical circuitry configured to broadcast a plant community user request. In an embodiment, the plant community exchange component 116 includes electrical circuitry configured to broadcast an offer to rescue a plant or have a plant rescued, an offer to adopt a plant or to place a plant for adoption, or an offer to sell a plant or to buy a plant. In an embodiment, the electrical circuitry configured to communicate a notice, broadcast an opportunity or the like includes one or more client devices, data communication systems, electronic communication systems, email systems, hubs, local operating systems, network operating systems, networks, receivers, routers, servers, switch, transmitters, transceivers, transmission systems, ecommerce systems the like.

In an embodiment, the plant community exchange component 116 includes electrical circuitry configured to broadcast an offer to buy, sell, or generate a virtual plant. In an embodiment, the plant community exchange component 116 includes electrical circuitry configured to broadcast an offer buy, sell, or generate a metaverse virtual plant. In an embodiment, the plant community exchange component 116 includes electrical circuitry configured to broadcast to one or more client devices the plant community user request. In an embodiment, the plant community exchange component 116 includes electrical circuitry configured to broadcast a plant community user request based on the generated one or more garden attributes or the generated one or more garden community recommendations. In an embodiment, the plant community exchange component 116 includes electrical circuitry configured to broadcast a user request to adopt a plant. In an embodiment, the plant community exchange component 116 includes electrical circuitry configured to broadcast a user request on one or more servers to sell a plant or to buy a plant.

In an embodiment, the plant community exchange component 116 includes electrical circuitry configured to broadcast a user request to rescue a plant or have a plant rescued. In an embodiment, the plant community exchange component 116 includes electrical circuitry configured to broadcast a user request to adopt a plant or to place a plant for adoption. In an embodiment, the plant community exchange component 116 includes electrical circuitry configured to broadcast a user request to an offer to sell a virtual plant. In an embodiment, the plant community exchange component 116 includes electrical circuitry configured to broadcast a user request to an offer to sell a metaverse virtual plant. In an embodiment, the plant community exchange component 116 includes electrical circuitry configured to broadcast a user request to an offer to sell a nonfungible token associated with a virtual plant.

In an embodiment, the plant community exchange component 116 includes electrical circuitry configured to electronically notify at least one member of plant community an availability of a plant for rescue. In an embodiment, the plant community exchange component 116 includes electrical circuitry configured to facilitate payment to a digital wallet responsive to one or more inputs indicative of transaction for a plant adoption. In an embodiment, the plant community exchange component 116 includes electrical circuitry configured to initiate an increase in value to a cryptocurrency wallet responsive to one or more inputs indicative of an acceptance to an offer to sell a plant. In an embodiment, the plant community exchange component 116 includes electrical circuitry configured to initiate a cryptocurrency transfer to or from a cryptocurrency wallet based on a plant community user transaction. In an embodiment, the plant community exchange component 116 includes electrical circuitry configured to facilitate a virtual payment to a seller for an offer to sell a plant based on one or more inputs indicative of a selection of a checkout URL associated with a virtual selling or purchasing cart. In an embodiment, the plant community exchange component 116 includes a graphical user interface display configured to present one or more instances of a virtual representation of the plant community user request.

Plant Wellness Potential Assessment Systems, Devices, and Methods

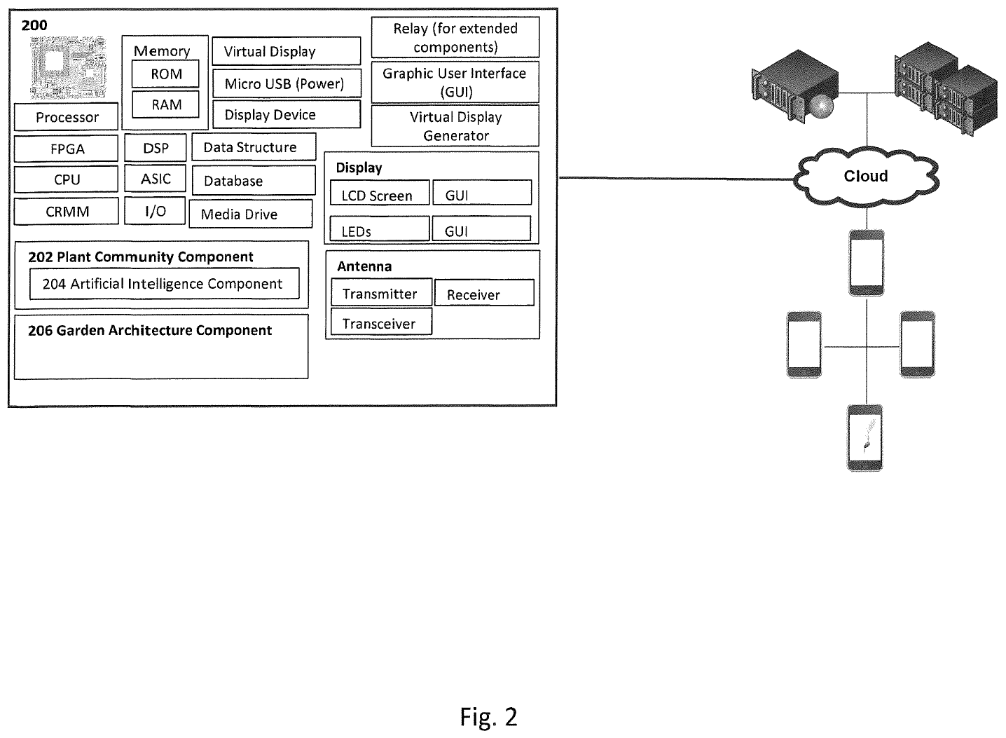

FIG. 2 show a wellness system 200 in which one or more of the disclosed methodologies or technologies can be implemented. In an embodiment, the wellness system 200 includes a plant assessment component 202 including electrical circuitry configured to evaluate one or more digital images of a plant, garden, or the like, and to determine a plant wellness potential profile based on a plant potential classifier trained to evaluate indicators of plant morphology and to predict a plant potential to improve health, elicit calmness, induce relaxation, increase alertness, mitigate fatigue, mitigate stress, or mitigate anxiety. In an embodiment, the wellness system 200 includes electrical circuitry having an plant potential analysis circuitry configured to perform a pixel by pixel analysis of one or more digital images of a plant and to determine a plant wellness potential profile based on a plant potential classifier trained to evaluate indicators of plant morphology and to predict a plant potential to improve health, elicit calmness, induce relaxation, increase alertness, mitigate fatigue, mitigate stress, or mitigate anxiety. In an embodiment, the wellness system 200 includes electrical circuitry having an plant potential analysis circuitry configured to employ computer vision techniques examine plant digital content and to determine a plant wellness potential profile based on computer vision technique classifier trained to identify, classify, evaluate, and the like indicators of plant morphology and to predict a plant potential to improve health, elicit calmness, induce relaxation, increase alertness, mitigate fatigue, mitigate stress, or mitigate anxiety. Non-limiting example of computer vision techniques includes depth perception, image analysis, image classification, image classification, image processing, instance segmentation, keypoint detection, object detection, object identification, object tracking, 3D object reconstruction, panoptic segmentation, object segmentation, semantic segmentation, and the like.

In an embodiment, the plant assessment component 202 includes an artificial intelligence component 204 trained to detect indicators of plant morphology and to determine the plant wellness potential profile based on threshold leaf size information, threshold leaf shape information, or leaf color information, and a wellness correlation policy. In an embodiment, the plant assessment component 202 includes an artificial intelligence component 204 trained to detect indicators of plant morphology and to determine the plant wellness potential profile based on leaf size percent distribution, leaf shape percent distribution, or leaf shape color percent distribution, or combinations thereof, and a plant morphology-plant wellness potential correlation policy. In an embodiment, the plant assessment component 202 includes an artificial intelligence component 204 for determining plant wellness potential comprising plant morphology and wellness correlation training sets. In an embodiment, the plant assessment component 202 includes an artificial intelligence component 204 that determines the plant wellness potential profile as function of plant morphology measures such as leaf size, leaf shape, leaf size distribution, leaf shape distribution, or leaf density.

In an embodiment, the wellness system 200 includes a garden architecture component 206 including electrical circuitry configured to generate one or more garden design recommendations based on at least one determined plant wellness potential. In an embodiment, the garden architecture component 206 includes electrical circuitry configured to generate the one or more garden design recommendations based on the at least one determined plant wellness potential, and at least one target space attribute. In an embodiment, the space attribute includes a space wellness attribute. In an embodiment, the space wellness attribute includes an anxiety mitigation space, a calming pace, a contemplation space, an energizing space, a fatigue mitigation space, a filtered air space, a health improvement space, a meditation space, a pollution abatement space, a relaxation space, or a stress mitigation space based on the at least one determined plant wellness potential, and at least one space wellness attribute. In an embodiment, the space wellness attribute includes a filtered air space, a health improvement space, or a pollution abatement space based on the at least one determined plant wellness potential, and at least one space wellness attribute. In an embodiment, the space attribute includes a physical space dimension or a physical space geometry. In an embodiment, the space attribute includes a metaverse space geometry, a metaverse space configuration, or metaverse access information. In an embodiment, the space attribute includes geolocation information for a location to determine environmental toxins information, pollutant information, air pollutant information, or exposome information.

In an embodiment, the garden architecture component 206 includes electrical circuitry configured to generate the one or more user-selectable virtual garden designs based on the at least one determined plant wellness potential, and at least one target space attribute. In an embodiment, the garden architecture component 206 includes electrical circuitry configured to generate the one or more garden design recommendations predicted to improve health, to elicit calmness, to induce relaxation, to increase alertness, to mitigate fatigue, to mitigate stress, to mitigate anxiety, or to mitigate pollution based on the at least one determined plant wellness potential, and at least one space wellness attribute.

In an embodiment, the garden architecture component 206 includes electrical circuitry configured to generate a space design recommendation predicted to improve health, to elicit calmness, to induce relaxation, to increase alertness, to mitigate fatigue, to mitigate stress, to mitigate anxiety, or to mitigate pollution, or combinations thereof. In an embodiment, the garden architecture component 206 includes electrical circuitry configured to generate the one or more garden design recommendations predicted to provide physical, spiritual, and emotional balance to the user.

In an embodiment, the one or more garden design recommendations include recommended plant species information, recommended plant physical dimension information, recommended rescue plants information, recommended plant adoption information, a recommended garden design to reduce fatigue including plants with small, curvy leaves, a recommended garden design to improve concentration including plants with small, straight leaves, a recommended garden design to increase energy including plants with large, curvy leaves, and the like.

In an embodiment, the garden architecture component 206 includes electrical circuitry including one or more graphical user interface that allow a user to select one or more garden plant morphology attributes and to genera a garden wellness potential based on the selected one or more garden plant morphology attributes. In an embodiment, the selected one or more garden plant morphology attributes include a leaf size percent distribution, a leaf shape percent distribution, a leaf shape color percent distribution, or combinations thereof.

Image Morphing Systems, Devices, and Methods

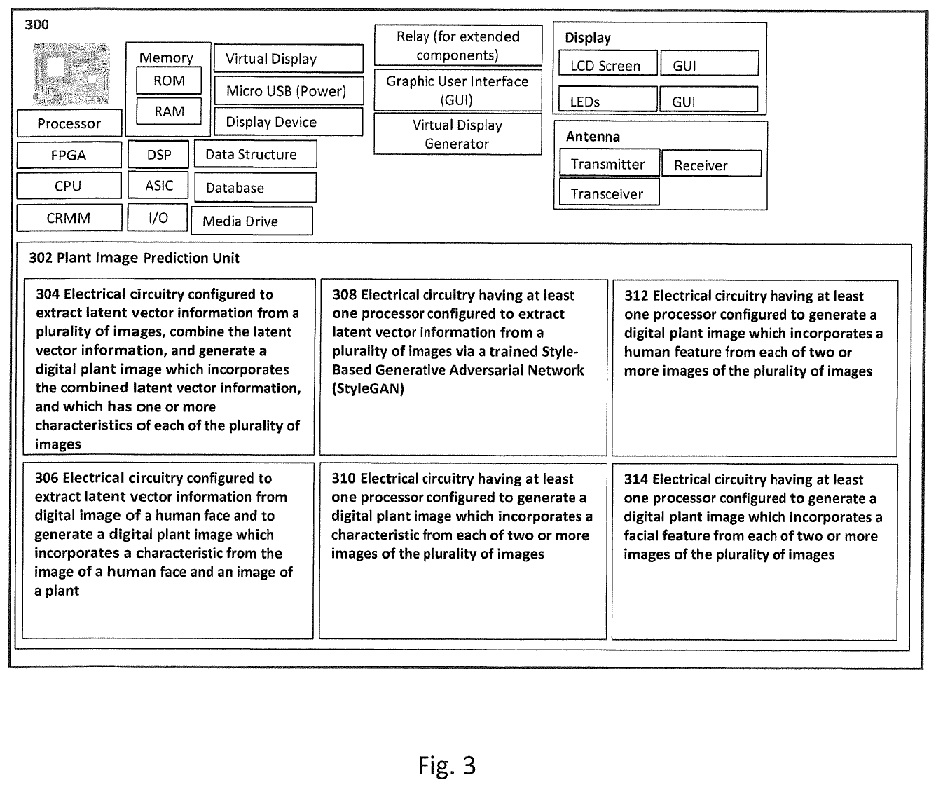

FIG. 3 shows a system 300 in which one or more of the disclosed methodologies or technologies can be implemented such as, for example, generating a digital plant, a digital garden, a virtual plant, a virtual garden, a plant or garden image, a plant or garden video, digital content including a plant or garden, and the like. In an embodiment, the system for generating a digital plant 300 includes a plant image prediction unit 302. In an embodiment, the plant image prediction unit 302 includes electrical circuitry 304 configured to extract latent vector information from a plurality of images, combine the latent vector information, and generate a digital plant image which incorporates the combined latent vector information, and which has one or more characteristics of each of the plurality of images. In an embodiment, the plant image prediction unit 302 includes electrical circuitry 306 configured to extract latent vector information from a digital image of a human face and to generate a digital plant image which incorporates a characteristic from the image of a human face and an image of a plant.

In an embodiment, the plant image prediction unit 302 comprises electrical circuitry 308 having at least one processor configured to extract latent vector information from a plurality of images via a trained Style-Based Generative Adversarial Network (StyleGAN). In an embodiment, the plant image prediction unit 302 comprises electrical circuitry 310 having at least one processor configured to generate a digital plant which incorporates a characteristic from each of two or more images of the plurality of images. In an embodiment, the plant image prediction unit 302 comprises electrical circuitry 312 having at least one processor configured to generate a digital plant which incorporates at least one human feature from each of two or more images of the plurality of images. In an embodiment, the plant image prediction unit 302 comprises electrical circuitry 314 having at least one processor configured to generate a digital plant which incorporates a facial feature from each of two or more images of the plurality of images.
Characteristics: shape, color, pose, facial feature, hair style,
Facial features: a face type, a facial feature (e.g., eye, lips, brow)
Human characteristics: personality.

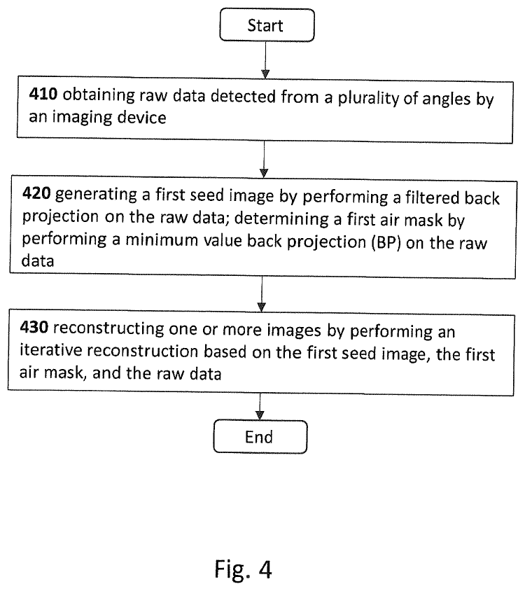

FIG. 4 is a flow diagram of a method 400 implemented on a machine having at least one processor and at least one storage device for constructing a digital plant image having one or more anthropomorphic quality. At 410 the method 400 includes obtaining raw data detected from a plurality of angles by an imaging device. At 420 the method 400 includes generating a first seed image by performing a filtered back projection on the raw data; determining a first air mask by performing a minimum value back projection (BP) on the raw data. At 430 the method 400 includes reconstructing one or more images by performing an iterative reconstruction based on the first seed image, the first air mask, and the raw data.

Plant Personification Systems, Devices, and Methods

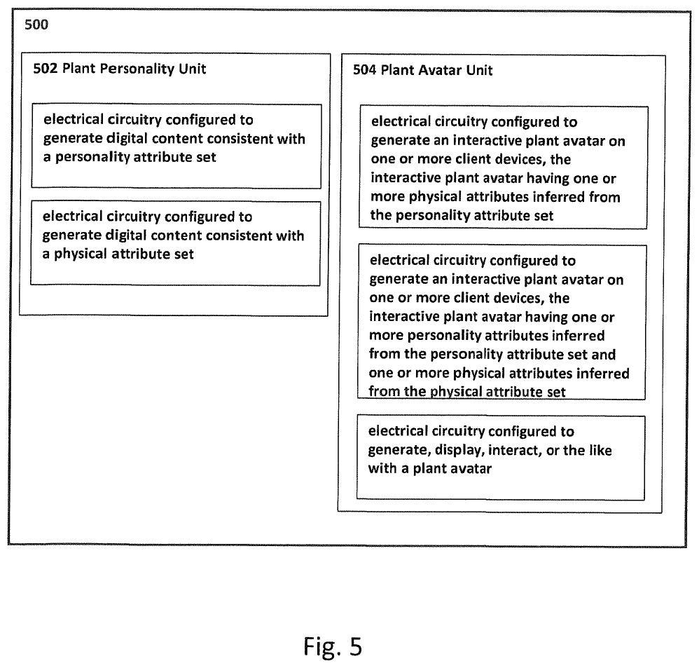

Referring to FIG. 5, in an embodiment, an interactive plant avatar system 500 includes a plant personality unit 502 including electrical circuitry configured to generate digital content consistent with a personality attribute set. In an embodiment, the personality unit 502 includes electrical circuitry configured to generate digital content consistent with a physical attribute set. In an embodiment, the interactive plant avatar system 500 includes a plant avatar unit 504 including electrical circuitry configured to generate an interactive plant avatar on one or more client devices, the interactive plant avatar having one or more physical attributes inferred from the personality attribute set. In an embodiment, the interactive plant avatar unit 504 includes electrical circuitry configured to generate an interactive plant avatar on one or more client devices, the interactive plant avatar having one or more personality attributes inferred from the personality attribute set and one or more physical attributes inferred from the physical attribute set. In an embodiment, the physical attribute set comprises a facial feature set. In an embodiment, the physical attribute set comprise a facial expressions attribute set. In an embodiment, the in physical attribute set comprise reference social trait facial images. In an embodiment, the physical attribute set comprises a shape of and the spacing around your eyes, nose, and mouth. See e.g., https://wgno.com/news/what-facial-features-do-people-look-at-first/In

an embodiment, the plant avatar unit 504 includes electrical circuitry configured to generate, display, interact, or the like with a plant avatar. In an embodiment, the plant avatar unit 504 is configured to generate an interactive plant avatar that reacts to a communication from a user via a user interface. For example, in an embodiment, the plant avatar unit 504 is configured to generate an interactive plant avatar that reacts to a communication from a user via a graphical user interface. In an embodiment, the plant avatar unit 504 is configured to generate an interactive plant avatar that reacts to voice cues or user gesturer cues. In an embodiment, the plant avatar unit 504 is configured to generate an interactive plant avatar configured to receive one or more gestures, text, touch, or voice inputs.

In an embodiment, the plant avatar unit 504 is configured to generate an interactive plant avatar configured to interact with a user via one or more automatic speech recognition protocols. See e.g., US Patent Application No. 20170310934 (the contents of which are incorporated herein by reference in their entirety). In an embodiment, the plant avatar unit 504 is configured to generate an interactive plant avatar configured to interact with a user via a human-computer dialog system using natural language. In an embodiment, the plant avatar unit 504 is configured to generate an interactive plant avatar configured to interact with a user via a Generative Pre-training Transformer (ChatGPT) component including circuitry configured to generate digital content including text, images, video, audio, synthetic data, or the like. In an embodiment, the plant avatar unit 504 is configured to generate an interactive plant avatar configured to interact with a user via one or more Markov chain processes. In an embodiment, the plant avatar unit 504 is configured to generate an interactive plant avatar configured to interact with a user via an artificial intelligence enabled prosses. In an embodiment, the plant avatar unit 504 is configured to generate an interactive plant avatar configured to interact with a user via conversational artificial process. In an embodiment, the plant avatar unit 504 is configured to generate an interactive plant avatar configured to interact with a user via deep Sequence-to-Sequence (Seq2seq) model having multiple layered of Long Short-Term Memory networks. see e.g., https://www.sciencedirect.com/science/article/pii/S2666827020300062

In an embodiment, the plant avatar unit 504 is configured to generate an interactive plant avatar configured to interact with a user using a chatbot process, a Generative Pre-trained Transformer process, and the like. See e.g., Eleni Adamopoulou, et al., Chatbots: History, technology, and applications, Machine Learning with Applications, Volume 2 (2020), 100006, https://doi.org/10.1016/j.mlwa.2020.100006; (https://www.sciencedirect.com/science/article/pii/S2666827020300062)

In an embodiment, the plant avatar unit 504 is configured to generate an interactive plant avatar configured to interact with a user using a linguistic based chatbot process. In an embodiment, the plant avatar unit 504 is configured to generate an interactive plant avatar configured to interact with a user using a rule-based chatbot process.

In an embodiment, the plant avatar unit 504 includes process circuitry configured to manage a linguistic or rules-based chatbots create conversational flows using if/then logic. In an embodiment, the plant avatar unit 504 includes process circuitry configured to manage a keyword recognition-based chatbots utilizing Natural Language Processing (NLP) to determine how to serve an appropriate response to the user. In an embodiment, the plant avatar unit 504 is operably coupled to a machine learning chatbot. In an embodiment, the machine learning chatbot with contextual awareness.

In an embodiment, the plant avatar unit 504 is configured to change at least one visual feature of the interactive plant avatar based on a user-specified change. In an embodiment, the plant avatar unit 504 includes electrical circuitry for allowing a user to select from a pre-defined menu of plant avatar changes or modification and for generating a control signal for actuation of a change at least one visual feature of the interactive plant avatar according to the selected change or modification from the pre-defined menu of plant avatar changes or modifications.

Aging Avatar, Evolving Avatars, Morphing Avatars, or the Like Systems, Devices, and Methods

In an embodiment, the plant avatar unit 504 includes electrical circuitry configured to change at least one visual feature of the interactive plant avatar based on a time-evolving protocol. In an embodiment, the plant avatar unit 504 is configured to change at least one visual feature of the interactive plant avatar based on a time-evolving plant aging protocol. In an embodiment, the plant avatar unit 504 is configured to change at least one visual feature of the interactive plant avatar based on an age-progression. In an embodiment, the plant avatar unit 504 is configured to change at least one visual feature of the interactive plant avatar based on an age-progression associate with a chronological age of a user.

In an embodiment, the plant avatar unit 504 is configured to vary an age indicator of the interactive plant avatar based on a digital age-progression process. In an embodiment, the plant avatar unit 504 is configured to change at least one audio feature of the interactive plant avatar based on a time-evolving program. In an embodiment, the plant avatar unit 504 is configured to change an audio response feature of the interactive plant avatar based on a time-evolving plant aging protocol. In an embodiment, the plant avatar unit 504 is configured to change an inflection, pitch, resonance, rhythm, tempo, texture, timber, tone, vocal fry, voice tremor, or volume of a plant audio response based a time-evolving plant aging protocol. In an embodiment, the plant avatar unit 504 is configured to generate an avatar on a display that mimics non-verbal cues including gestures, postures, movements, facial expressions, or the like. See e.g., U.S. Patent Publication No. 2017/0310934. In an embodiment, the plant avatar unit 504 includes electrical circuitry configured to change, modify, predict, determine at least one plant trait based on a user-specified personality plant personality attribute or trait.

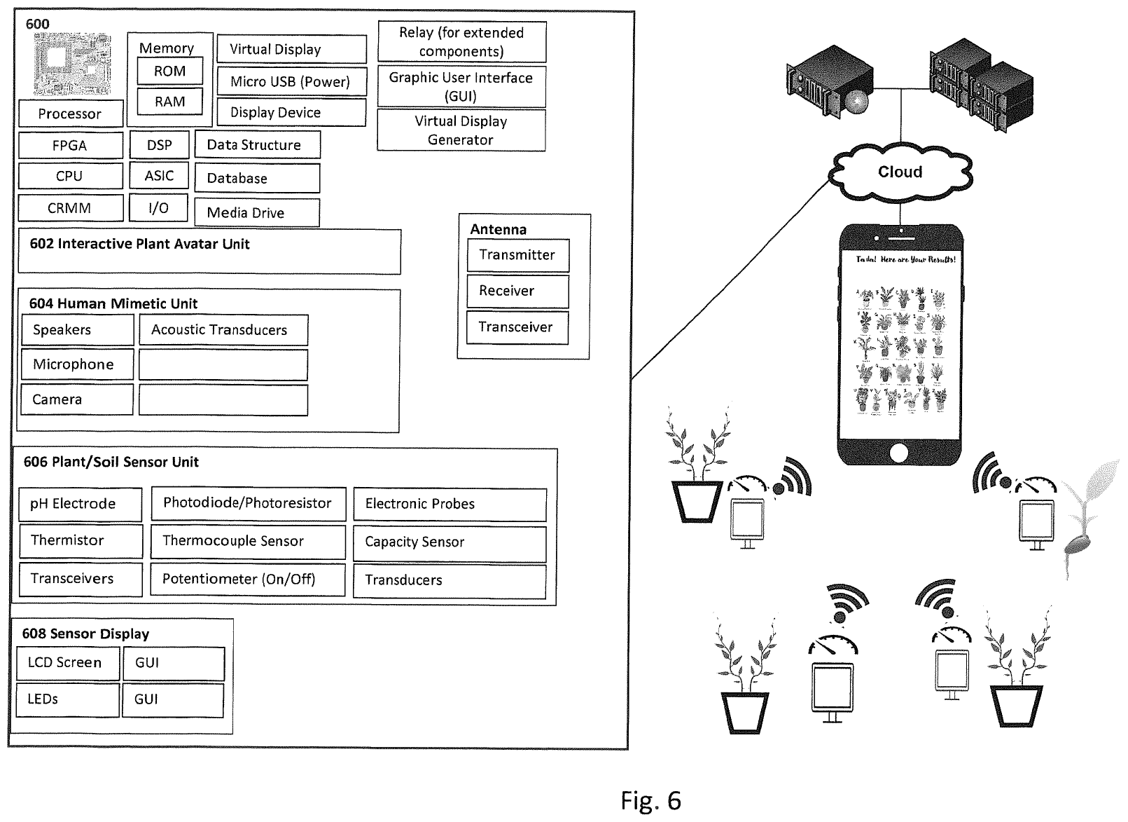

FIGS. 6 and 12-27 show a system 600 in which one or more of the disclosed methodologies or technologies can be implemented. In an embodiment, the system 600 includes an Interactive Plant Avatar Unit 602 including processing circuitry configured to generate an interactive plant avatar on one or more client devices, the interactive plant avatar having one or more physical attributes inferred from a physical attribute set and one or more personality attributes inferred from a personality attribute set.

In an embodiment, the system 600 includes a Human Mimetic Unit 604 including processing circuitry configured to generate physical attributes or personality attributes. In an embodiment, Human Mimetic Unit 604 including processing circuitry configured to generate at least one of a set of physical attributes or a set of personality attributes base on one or more inputs from sensor associated with a plant and indicative a soil or plant status.

In an embodiment, the system 600 includes a Plant or Soil Sensor Unit 606 configured to collect sensor data regarding a plant status. In an embodiment, the Plant or Soil Sensor Unit 606 is operably coupled to one or more sensor configured to measure, detect, assess, etc., soil, plant, or environmental information.

Non-limiting examples of sensor include capacitance sensors, dielectric sensors, environmental sensors, light meters, moisture meters, moisture sensor, nodes, opacity sensors, pH meters, pH sensors, pollution sensors, soil moisture meters, sunlight exposure sensor, temperature sensor, thermocouples, transducers, Bluetooth enabled sensors, wireless enabled sensors, or the like.

In an embodiment, the system 600 includes a Sensor Display 608. In an embodiment, the system 600 includes at least one hardware processor and a memory storing instructions that, when executed by the at least one hardware processor, cause the system to perform operations comprising generate physical attribute set and a personality attribute set; and generate an interactive plant avatar on one or more client devices, the interactive plant avatar having one or more physical attributes inferred from the physical attribute set and one or more personality attributes inferred from the personality attribute set.

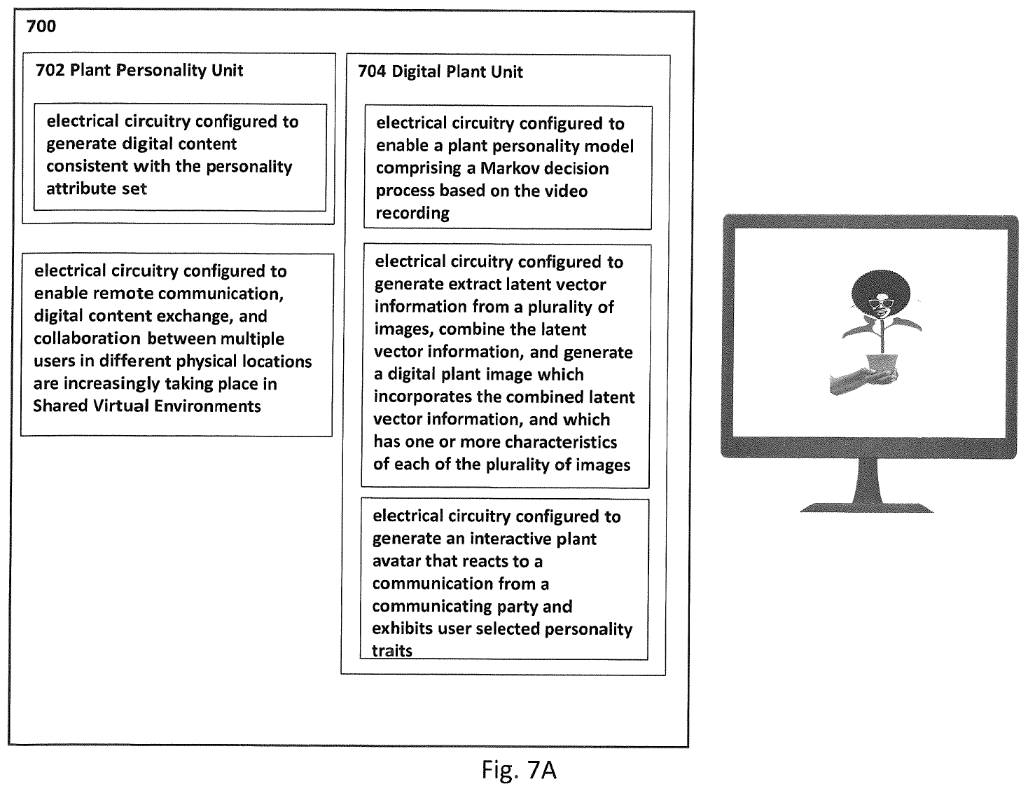

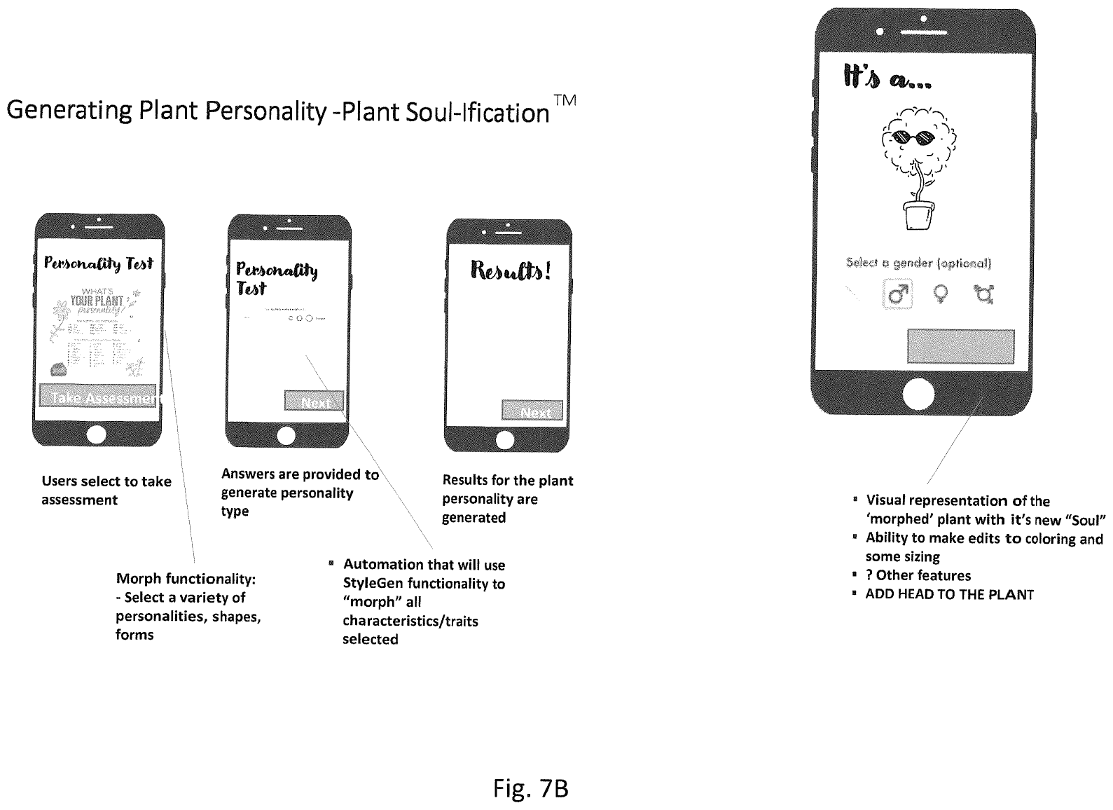

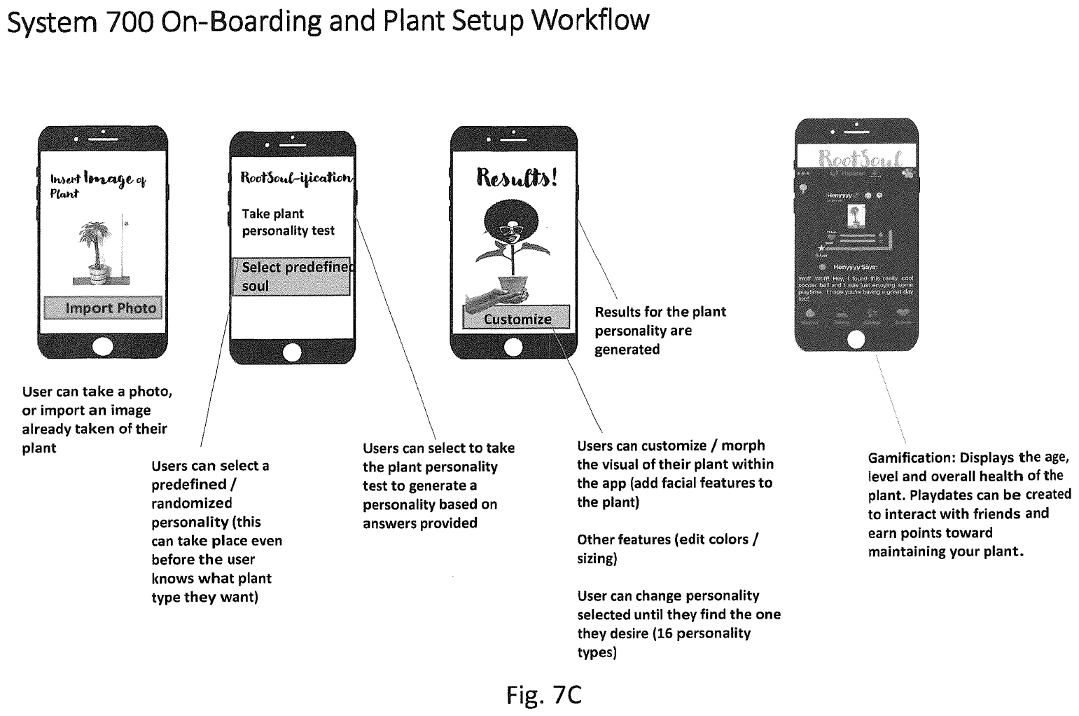

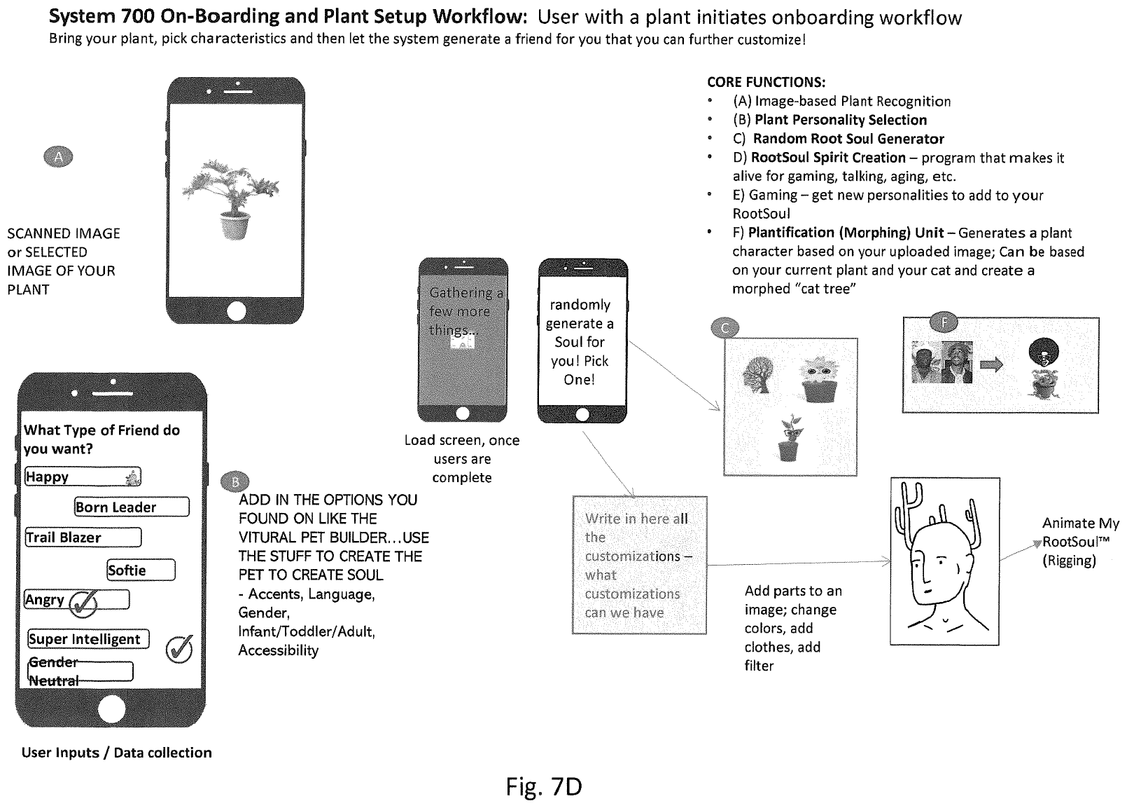

Referring to FIGS. 7A-7D, in an embodiment, the system 700 for generating a digital plant includes a plant personality unit 702 including electrical circuitry configured to generate digital content consistent with the personality attribute set.

In an embodiment, the system 700 for generating a digital plant includes a digital plant unit 704 including electrical circuitry configured to generate a digital plant. In an embodiment, the digital plant unit 704 includes electrical circuitry configured to enable a plant personality model comprising a Markov decision process based on the video recording. In an embodiment, the digital plant unit 704 includes electrical circuitry configured to generate extract latent vector information from a plurality of images, combine the latent vector information, and generate a digital plant image which incorporates the combined latent vector information, and which has one or more characteristics of each of the plurality of images. In an embodiment, the digital plant unit 704 includes electrical circuitry configured to generate an interactive plant avatar that reacts to a communication from a communicating party and exhibits user selected personality traits.

In an embodiment, the system 700 for generating a digital plant includes electrical circuitry configured to enable remote communication, digital content exchange, and collaboration between multiple users in different physical locations are increasingly taking place in Shared Virtual Environments. See e.g., U.S. Pat. No. 11,100,561 (the contents of which are incorporated herein by reference in their entirety).

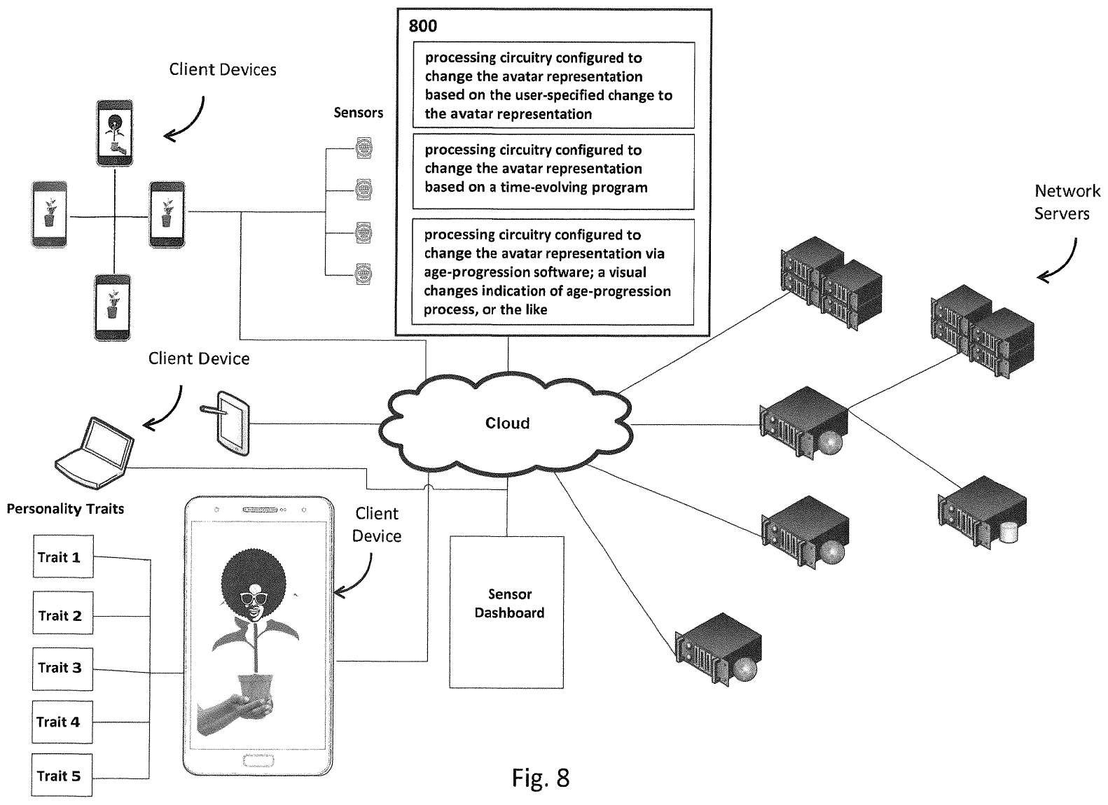

FIG. 8 shows a system 800. In an embodiment, a system 800 includes at least one hardware processor; and a memory storing instructions that, when executed by the at least one hardware processor cause the system 800 to perform operations comprising: generating an avatar representation of a plant responsive to one or more inputs; changing the avatar representation responsive to a time-evolution protocol; changing the avatar representation based on the user-specified change to the avatar representation; determining a current status of the user based on real-time data obtained from a first client device associated with the user; generating an updated avatar representation based on the current status of the user; and causing one or more second client devices to display the updated avatar in association with a communication of the user.

In an embodiment, a system 800 includes processing circuitry configured to change the avatar representation based on the user-specified change to the avatar representation. In an embodiment, a system 800 includes processing circuitry configured to change the avatar representation based on a time-evolving program. In an embodiment, a system 800 includes processing circuitry configured to change the avatar representation via age-progression software; a visual changes indication of age-progression process, or the like. See. E.g., https://www.washington.edu/news/2014/04/09/see-what-a-child-will-look-like-using-automated-age-progression-software/. In an embodiment, a system 800 includes processing circuitry configured to render an avatar representation of the user by inferring visually exemplifiable user characteristics corresponding to the user based on an analysis of the user data. In an embodiment, the user characteristics comprises an inferred physical trait and the inferred personality trait physical trait and a personality trait. In an embodiment, a system 800 includes electrical circuitry configured to generate the avatar representation of the user based on the inferred physical trait and the inferred personality trait.

Metaverse Systems, Devices, and Methods

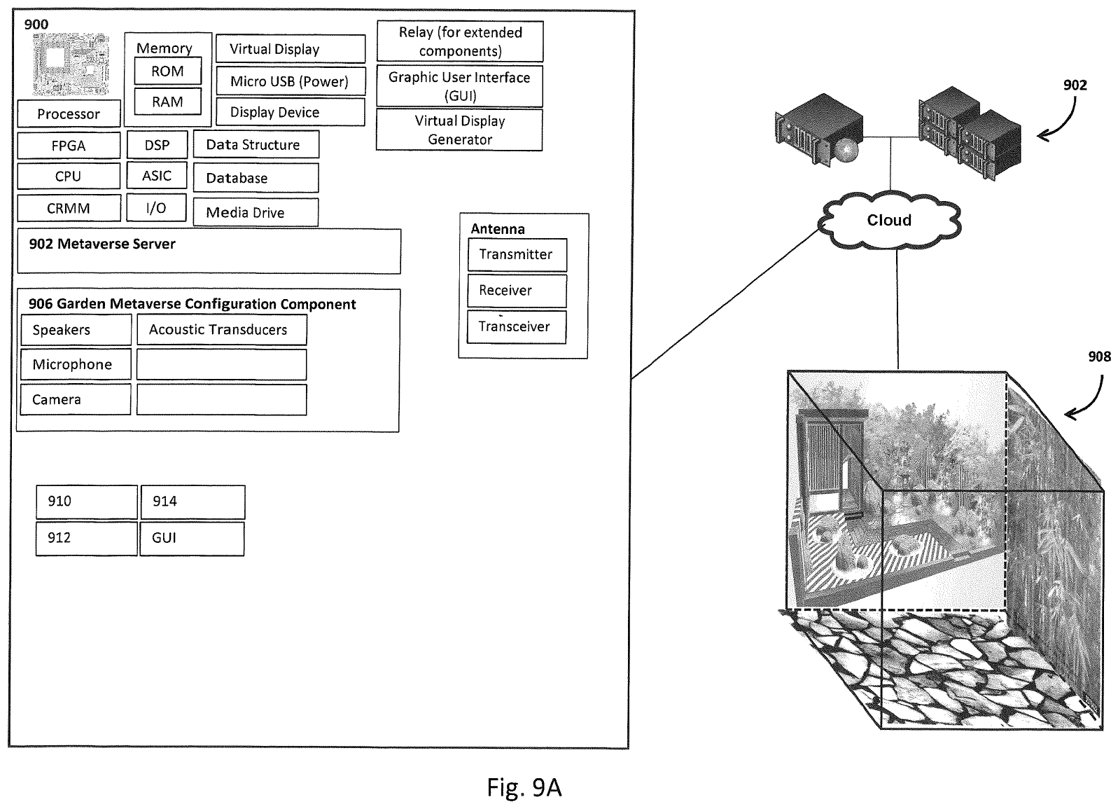

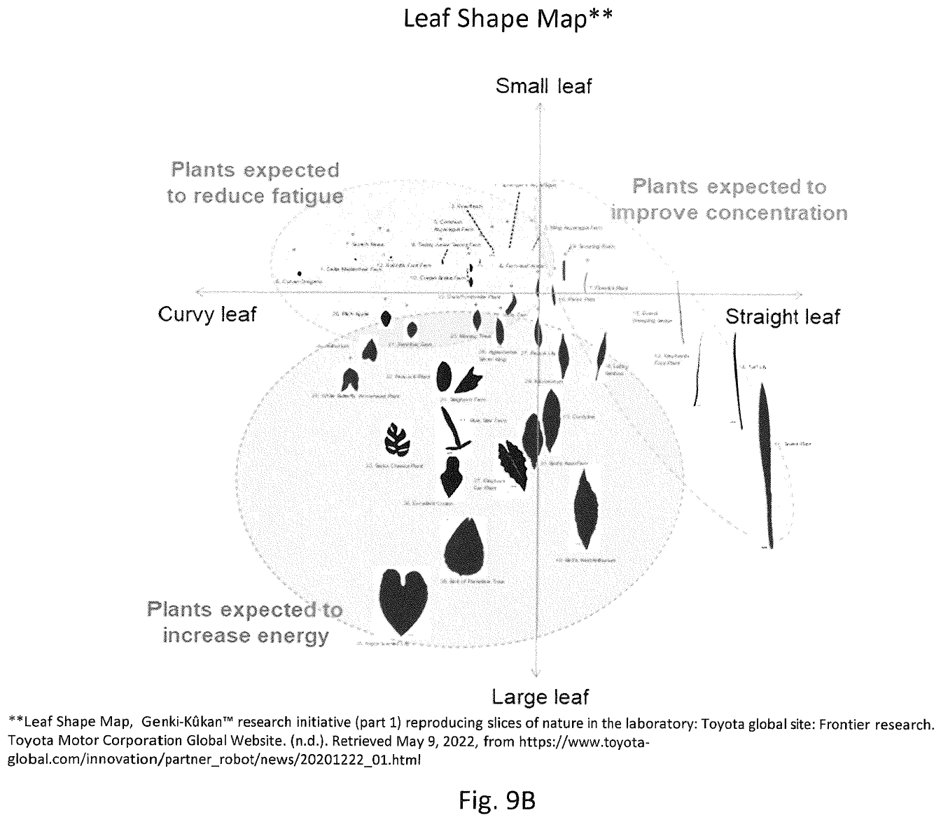

FIG. 9A shows a metaverse system 900 in which one or more of the disclosed methodologies or technologies can be implemented, such as, for example, a metaverse client terminal for providing a metaverse space capable of enabling interaction between users in a plant community. In an embodiment, the metaverse system 900 includes a metaverse server 902 coupled to a client computer 904.

In an embodiment, the metaverse system 900 includes a garden metaverse configuration component 906 is configured to generate a metaverse 908 including one or more virtual plants having a geometric shape based on one or more inputs indicative of a wellness attribute. In an embodiment, the wellness attribute comprises an anxiety mitigation space, a calming pace, a contemplation space, an energizing space, a fatigue mitigation space, a filtered air space, a health improvement space, a meditation space, a pollution abatement space, a relaxation space, or a stress mitigation space based on the at least one determined plant wellness potential, and at least one space wellness attribute. In an embodiment, the metaverse 908 is accessible by avatars of users that access the metaverse via client devices.

In an embodiment, the garden metaverse configuration component 906 is configured to generate a metaverse including one or more virtual plants having a plant morphology including a leaf size, a leaf shape, a leaf color, a leaf size percent distribution, a leaf shape percent distribution, or leaf shape color percent distribution, or combinations thereof based on the one or more inputs indicative of the wellness attribute.

In an embodiment, the garden metaverse configuration component 906 is configured to generate a metaverse including one or more virtual plants having a plant morphology including a leaf size, a leaf shape, a leaf color, a leaf size percent distribution, a leaf shape percent distribution, or leaf shape color percent distribution, or combinations thereof based on a plant morphology-plant wellness potential correlation policy. In an embodiment, the garden metaverse configuration component 906 is further configured to generate one or more objects within the garden metaverse having a geometric shape that mimic a plant attribute associated with the wellness attribute. In an embodiment, the garden metaverse configuration component 906 is configured to generate a metaverse including one or more virtual plants having a plant morphology including a leaf size, a leaf shape, a leaf color, a leaf size percent distribution, a leaf shape percent distribution, or leaf shape color percent distribution, or combinations thereof based on at least one input indicative of a target garden characteristic and at least one input indicative of the predicted calmness state.

In an embodiment, the wellness metaverse system 900 includes electrical circuitry 910 including a hardware processor, a display, and a user interface configured to render and access a wellness metaverse. Non-limiting examples of a user interface include a virtual display, an interactive virtual display, a user interface, a command line user interface, a menu driven user interface, a graphical user interface, a display, a user input device, a system of interactive visual components, and the like.

In an embodiment, the wellness metaverse system 900 includes electrical circuitry 912 including a hardware processor, a display, and a user interface configured to render and access a menu of selectable room attributes rendered on the display of a room within the wellness metaverse. In an embodiment, a user operates the user interface to navigate to a garden metaverse space.

In an embodiment, the wellness metaverse system 900 includes electrical circuitry 912 including a hardware processor, a display, and a user interface configured to allow a user to select one or more garden plant morphology attributes and to genera a garden wellness potential based on the selected one or more garden plant morphology attributes. In an embodiment, the

In an embodiment, the wellness metaverse system 900 includes electrical circuitry 912 including a hardware processor, a display, and a user interface configured render and access a wellness metaverse that includes digital content that is based on the selected one or more garden plant morphology attributes include a leaf size percent distribution, a leaf shape percent distribution, a leaf shape color percent distribution, or combinations thereof.

For example, in an embodiment the rendered metaverse includes mosaics having geometric patterns, designs, shapes that mimic a plant geometric attribute associated with the wellness attribute. In the embodiment, the rendered metaverse includes mosaics having objects that mimic small curvy leaves based on a wellness attribute associated with fatigue. In the embodiment, the rendered metaverse includes mosaics having objects that mimic large curvy leaves based on a wellness attribute associated with a user energy level. In the embodiment, the rendered metaverse includes mosaics having objects that mimic small straight, leaves based on a wellness attribute associated with concentration. In the embodiment, the rendered metaverse includes mosaics having objects that mimic an admixture of mimic small curvy leaves based on a wellness attribute associated with fatigue, objects that mimic large curvy leaves based on a wellness attribute associated with a user energy level, and objects that mimic small straight, leaves based on a wellness attribute associated with concentration.

Referring to FIGS. 9A and 9B, in an embodiment, the wellness metaverse system 900 includes electrical circuitry 914 including a machine learning component configured to classifying plants in part in accordance with leaf shape map protocol such as those descried in Japanese Patent Publication No. JP2022019372A (the contents of which are incorporated herein by reference in their entirety).

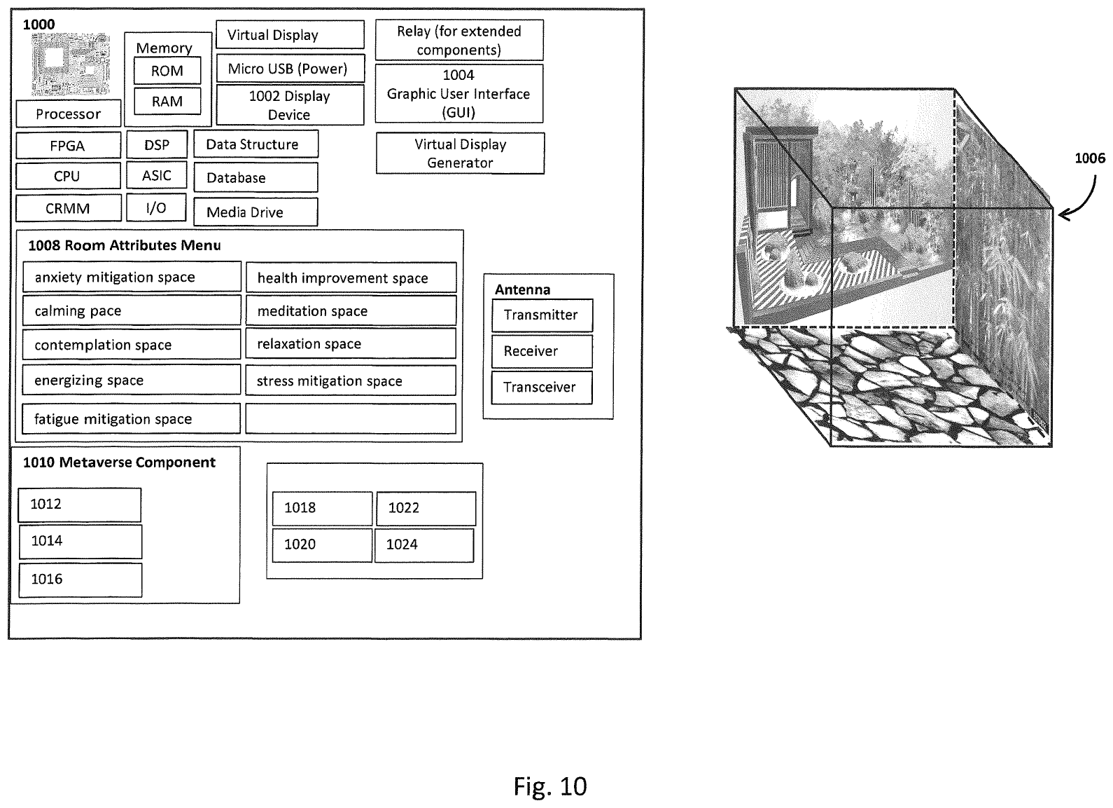

FIG. 10 shows a wellness metaverse system 1000 in which one or more of the disclosed methodologies or technologies can be implemented. In an embodiment, the wellness metaverse system 1000 includes electrical circuitry including a hardware processor, a display 1002, and a graphical user interface (GUI) 1004 configured to render and access a wellness metaverse 1006. In the embodiment, the GUI includes a menu 1008 of selectable room attributes rendered on the display of a room within the wellness metaverse. In an embodiment, a user operates the GUI 1004 to navigate to a garden metaverse space. In an embodiment, the menu 1008 of selectable room attributes include at least one of an anxiety mitigation space, a calming pace, a contemplation space, an energizing space, a fatigue mitigation space, a health improvement space, a meditation space, a relaxation space, or a stress mitigation space.

In the embodiment, a metaverse component 1010 includes electrical circuitry 1012 configured to generate virtual plants within garden metaverse having a wellness attribute that corresponds to a selectable room attribute.

In the embodiment, the metaverse component 1010 includes electrical circuitry 1014 configured to generate virtual plants within garden metaverse having a leaf size, a leaf shape, a leaf color, or a leaf size distribution that corresponds to a selectable room attribute.

In the embodiment, the metaverse component 1010 includes electrical circuitry 1016 configured to generate virtual plants within garden metaverse having a leaf size, a leaf shape, a leaf color, a leaf size percent distribution, a leaf shape percent distribution, or leaf shape color percent distribution, or combinations thereof based on at least one input indicative of a target garden characteristic and at least one input indicative of a wellness attribute.

In the embodiment, the wherein the GUI 1004 includes a map overview of an entire garden metaverse.

In an embodiment, the wellness metaverse system 1000 includes electrical circuitry 1018 including a hardware processor, a display, and a graphical user interface (GUI) configured to render on a display a virtual room within the metaverse having a plurality of virtual plants having a plant morphology including a leaf size, a leaf shape, a leaf color, a leaf size percent distribution, a leaf shape percent distribution, or leaf shape color percent distribution, or combinations thereof rendered based on the one or more inputs indicative of the wellness attribute.

In an embodiment, the wellness metaverse system 1000 includes electrical circuitry 1020 including a hardware processor, a display, and a graphical user interface (GUI) configured to rendered on the display is a virtual room within the metaverse having a plurality of virtual objects within the garden metaverse having a geometric shape that mimic a plant attribute associated with the wellness attribute. In the embodiment, rendered on the display is a virtual room within the metaverse having a plurality of virtual objects within the garden metaverse that mimic small curvy leaves based on a wellness attribute associated with fatigue.

In an embodiment, the wellness metaverse system 1000 includes electrical circuitry 1022 including a hardware processor, a display, and a graphical user interface (GUI) configured to rendered on the display is a virtual room within the metaverse having a plurality of virtual objects within the garden metaverse that mimic small straight, leaves based on a wellness attribute associated with concentration. In the embodiment, rendered on the display is a virtual room within the metaverse having a plurality of virtual objects within the garden metaverse that mimic large curvy leaves based on a wellness attribute associated with a user energy level.

In an embodiment, the wellness metaverse system 1000 includes electrical circuitry 1024 including a hardware processor, a display, and a graphical user interface (GUI) configured to give users the physical sensation of being in another place and sharing it with other people.

In an embodiment, the wellness metaverse system 1000 includes a controller or other piece of hardware that enable users to interact with the online metaverses. In an embodiment, the wellness metaverse system 1000 includes electrical circuitry configured to enable a user experience that is partially real life, partially digital running in parallel with the physical world. In an embodiment, the wellness metaverse system 1000 includes controller, hardware to render metaverse, grant access, enable user experience, to allow a user to navigate within a metaverse in the cloud with a full-body avatar, to detect physical world variables, to enable directional sound, or the like.

In an embodiment, the wellness metaverse system 1000 includes an Interactive hardware controller. In an embodiment, the wellness metaverse system 1000 includes electrical circuitry including a hardware processor, a display, and a graphical user interface (GUI) configured to connect user to an assistant that guides a plant rescue experience. In an embodiment, the wellness metaverse system 1000 includes electrical circuitry including a hardware processor, a display, and a graphical user interface (GUI) configured to connect user to an assistant that guides a home garden design. In an embodiment, the wellness metaverse system 1000 includes electrical circuitry including a hardware processor, a display, and a graphical user interface (GUI) configured to connect user to a plant community-improving their current cultivations; connecting them with a tight-knit community sharing common plant-related interests; and help to strengthen/personalize the relationship between a caretaker and their plant. In an embodiment, the wellness metaverse system 1000 includes electrical circuitry including a hardware processor, a display, and a graphical user interface (GUI) configured to connect user to a marketplace for plant collecting and purchasing plant-related products that needs a centralized hub to connect buyers with sellers and enthusiasts. In an embodiment, the wellness metaverse system 1000 includes electrical circuitry including a hardware processor, a display, and a graphical user interface (GUI) configured to enable an educational experience that introduces new climate activists to the sustainable hobby of gardening and the importance of environmental sustainability.

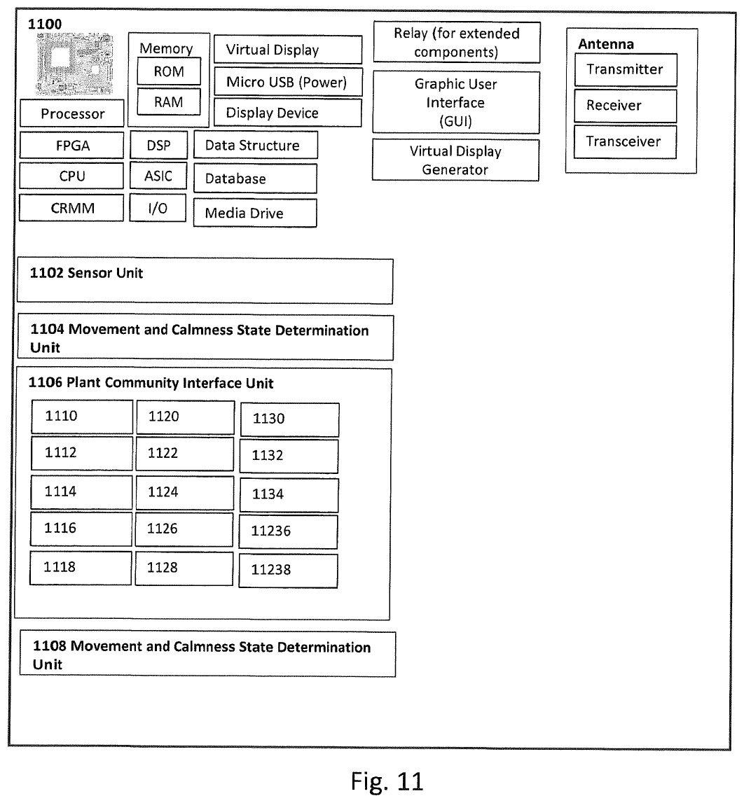

FIG. 11 shows a metaverse client terminal 1100 for providing a metaverse space capable of enabling interaction between users in a plant community in which one or more of the disclosed methodologies or technologies can be implemented. In an embodiment, the metaverse client terminal 1100 for providing a metaverse space capable of enabling interaction between users in a plant community includes a user sensor unit 1102 configured to collect sensor data regarding position, motion, location, biometrics, plant recognition/identification, user recognition/identification, and the like of at least a first user.

In an embodiment, the user sensor unit 1102 is configured to collect sensor data from one or remote sensors. I In an embodiment, the user sensor unit 1102 is configured to collect sensor data from one or more sensors operable to detect (e.g., assess, calculate, evaluate, determine, gauge, measure, monitor, quantify, resolve, sense, or the like) position, motion, location, biometrics, plant recognition/identification, user recognition/identification, and the like. Non-limiting examples of sensors include acoustic sensors, optical sensors, electromagnetic energy sensors, image sensors, photodiode arrays, charge-coupled devices (CCDs), complementary metal-oxide-semiconductor (CMOS) devices, transducers, optical recognition sensors, infrared sensors, radio frequency components sensors, thermo sensor, or the like.

In an embodiment, the user sensor unit 1102 is configured to collect sensor data from one or more sensors operable to determine user recognition/identification by facial or image recognition. For example, in an embodiment, the user sensor unit 1102 includes electrical circuitry operably coupled to one or more sensor (e.g., optical sensors, cameras, radiofrequency sensors, three-dimensional sensors (e.g., 3-D sensors operable to capture information about the shape of a face, etc.) or the like) operable to acquire image data. In an embodiment, during operation, the user sensor unit 1102 determines users' identity by detecting and analyzing distinct features of a user's face surface (e.g., structural features of the eye sockets, chin, nose, etc.). In an embodiment, the user sensor unit 1102 comprises three-dimensional sensor-based face recognition modalities. For example, in an embodiment, the user sensor unit 1102 includes one or more infrared light sensor operable to measures depth, position, motion, or the like. In an embodiment, the user sensor unit 1102 comprises a biometric sensor for identifying persons, objects, plants, and the like.

Non-limiting examples of location sensors include motion sensors, position sensors, global positioning sensor, inclinometers, magnetometers, micro electro-mechanical systems (MEMS), wearable sensors, and the like. Further non-limiting examples of location sensor, motion sensors, position sensors, and the like can be found in U.S. Pat. No. 10,314,733.

In an embodiment, the user sensor unit 1102 is configured to collect sensor data from one or more sensors operable to determine plant recognition/identification by image recognition. In an embodiment, the metaverse client terminal 1100 for providing a metaverse space capable of enabling interaction between users in a plant community includes a movement and calmness state determination unit 1104 configured to determine position information, motion information, biometric information, or calmness state information of the first user based on one or more inputs form the user sensor unit 1102.

In an embodiment, the metaverse client terminal 1100 for providing a metaverse space capable of enabling interaction between users in a plant community includes a plant community interface unit 1106 configured to transmit the position information, motion information, or biometric information, or calmness state information of the first user to a metaverse server, and to receive metaverse information data used to construct a metaverse space including the first user and to generate a first avatar moving in concurrence with the motion of the first user and displaying at least one indicator of calmness state of the first user.

In an embodiment, the movement and calmness state determination unit 1104 is configured to determine position information, motion information, biometric information, or calmness state information of at least a second user based on one or more inputs associated position sensor data, motion sensor data, biometric sensor data, or calmness state data of a second user, and to receive metaverse information data used to construct a metaverse space including the second user and to generate a second avatar moving in concurrence with the motion of the second user and displaying at least one indicator of calmness state of the second user. In an embodiment, the avatar of second user occupies space different from that of the avatar of the first user withing the metaverse.

In an embodiment, the plant community interface unit 1106 is configured to enable multiple users in different physical locations to adopt virtual plants within a shared metaverse space via interaction between corresponding avatars. In an embodiment, the plant community interface unit 1106 is configured to enable multiple users in different physical locations to be authenticated, authorized, and enabled for forming smart contracts associated with an adopt virtual plants within a within a shared metaverse space. In an embodiment, the plant community interface unit 1106 is configured to electronically notify at least one member of plant community an availability of a virtual or real plant for rescue.

In an embodiment, the plant community interface unit 1106 is configured to facilitate payment to a digital wallet responsive to one or more inputs indicative of transaction for a plant adoption. In an embodiment, the plant community interface unit 1106 is further configured to transmit plant data and to receive metaverse information data used to construct a metaverse space including a first plant avatar. In an embodiment, the plant community interface unit 1106 includes electrical circuitry 1108 configured to broadcast within the metaverse space an offer to rescue a plant or have a plant rescued, an offer to adopt a plant or to place a plant for adoption, or an offer to sell a plant or to buy a plant.

In an embodiment, the plant community interface unit 1106 includes electrical circuitry 1110 configured to broadcast within the metaverse space an offer to buy, sell, or generate a virtual plant. In an embodiment, the plant community interface unit 1106 includes electrical circuitry 1112 configured to broadcast within the metaverse space an offer buy, sell, or generate a metaverse virtual plant. In an embodiment, the plant community interface unit 1106 includes electrical circuitry 1114 configured to broadcast to one or more client devices the plant community user request.

In an embodiment, the plant community interface unit 1106 includes electrical circuitry 1116 configured to broadcast within the metaverse space a plant community user request based on the generated one or more garden attributes or the generated one or more garden community recommendations. In an embodiment, the plant community interface unit 1106 includes electrical circuitry 1118 configured to broadcast within the metaverse space a user request to adopt a plant. In an embodiment, the plant community interface unit 1106 includes electrical circuitry 1120 configured to broadcast within the metaverse space a user request to sell a plant or to buy a plant.

In an embodiment, the plant community interface unit 1106 includes electrical circuitry 1122 configured to broadcast within the metaverse space a user request to rescue a plant or have a plant rescued. In an embodiment, the plant community interface unit 1106 includes electrical circuitry 1124 configured to broadcast within the metaverse space a user request to adopt a plant or to place a plant for adoption. In an embodiment, the plant community interface unit 1106 includes electrical circuitry 1126 configured to broadcast within the metaverse space a user request to an offer to sell a virtual plant.

In an embodiment, the plant community interface unit 1106 includes electrical circuitry 1128 configured to broadcast within the metaverse space a user request to an offer to sell a metaverse virtual plant. In an embodiment, the plant community interface unit 1106 includes electrical circuitry 1130 configured to broadcast within the metaverse space a user request to an offer to sell a nonfungible token associated with a virtual plant. In an embodiment, the plant community interface unit 1106 includes electrical circuitry 1132 configured to electronically notify at least one member of plant community within the metaverse space an availability of a plant for rescue.

In an embodiment, the plant community interface unit 1106 includes electrical circuitry 1134 configured to facilitate payment to a digital wallet responsive to one or more inputs indicative of transaction for a plant adoption. In an embodiment, the plant community interface unit 1106 includes electrical circuitry configured to initiate an increase in value to a cryptocurrency wallet responsive to one or more inputs indicative of an acceptance to an offer to sell a plant.

In an embodiment, the plant community interface unit 1106 includes electrical circuitry 1136 configured to initiate a cryptocurrency transfer to or from a cryptocurrency wallet based on a plant community user transaction. In an embodiment, the plant community interface unit 1106 includes electrical circuitry 1138 configured to facilitate a virtual payment to a seller for an offer to sell a plant based on one or more inputs indicative of a selection of a checkout URL associated with a virtual selling or purchasing cart. In an embodiment, the plant community interface unit 1106 includes a graphical user interface display configured to present one or more instances of a virtual representation of the plant community user request.

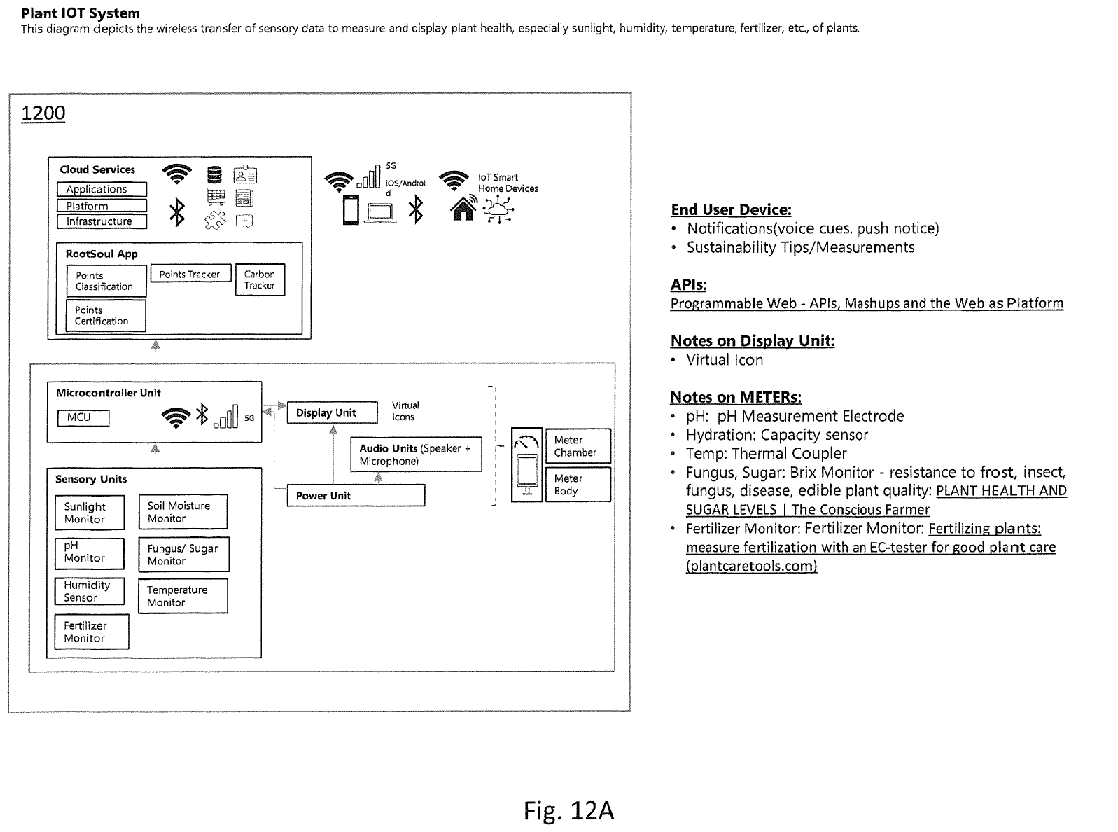

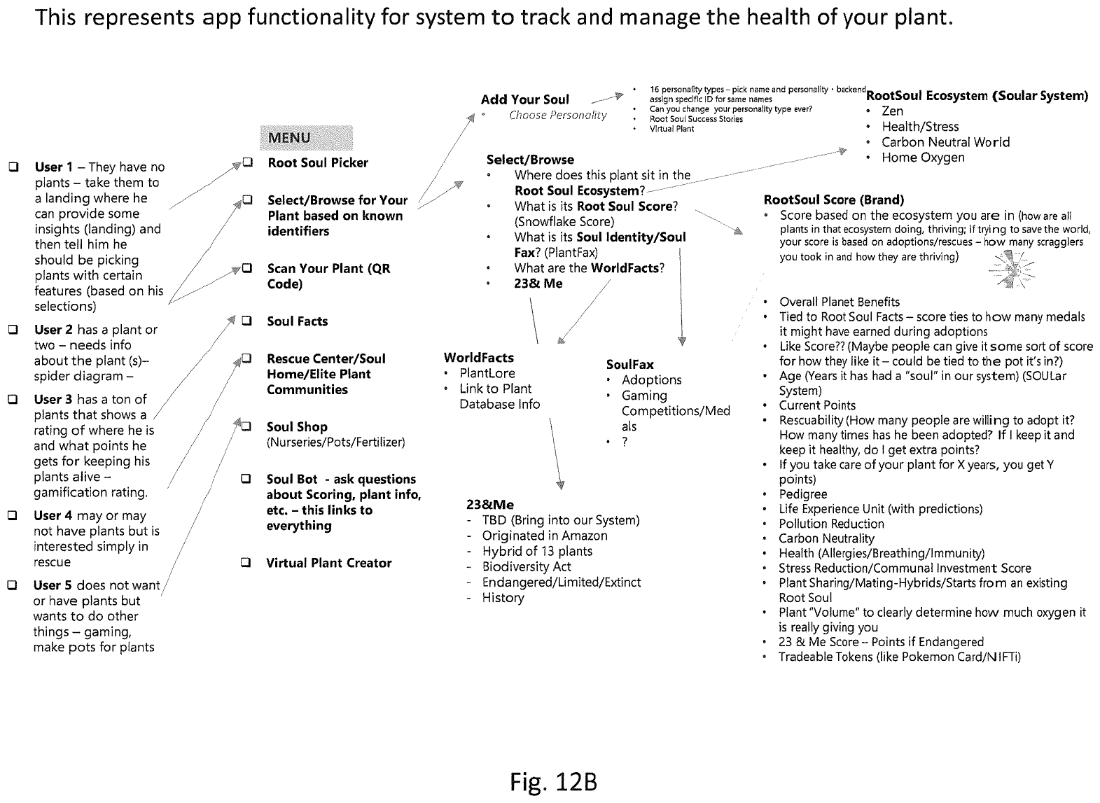

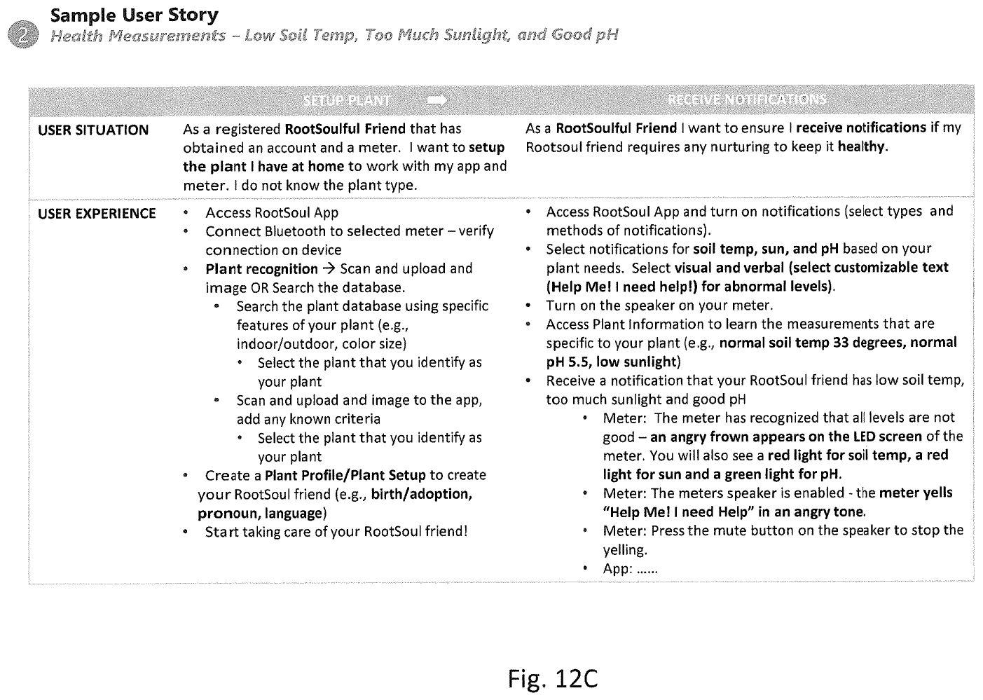

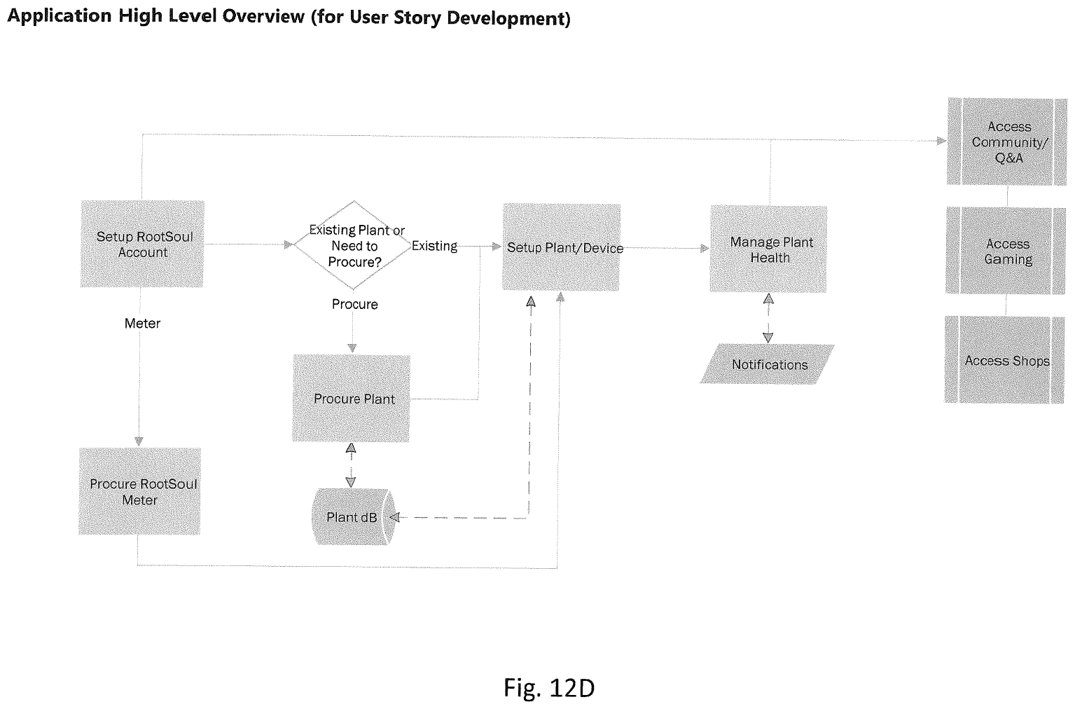

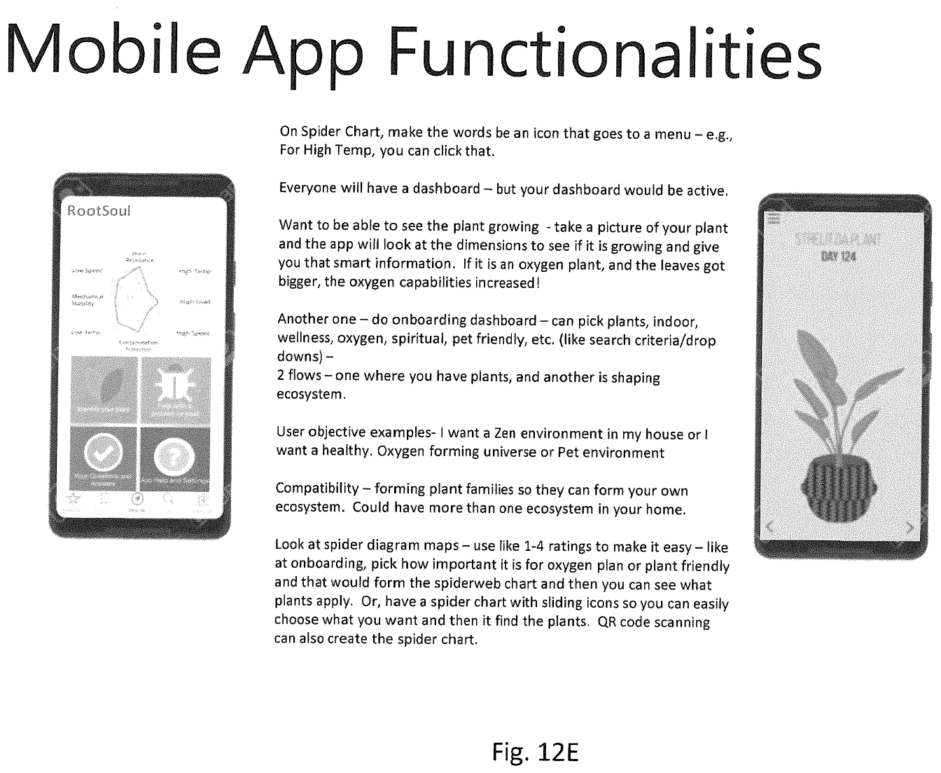

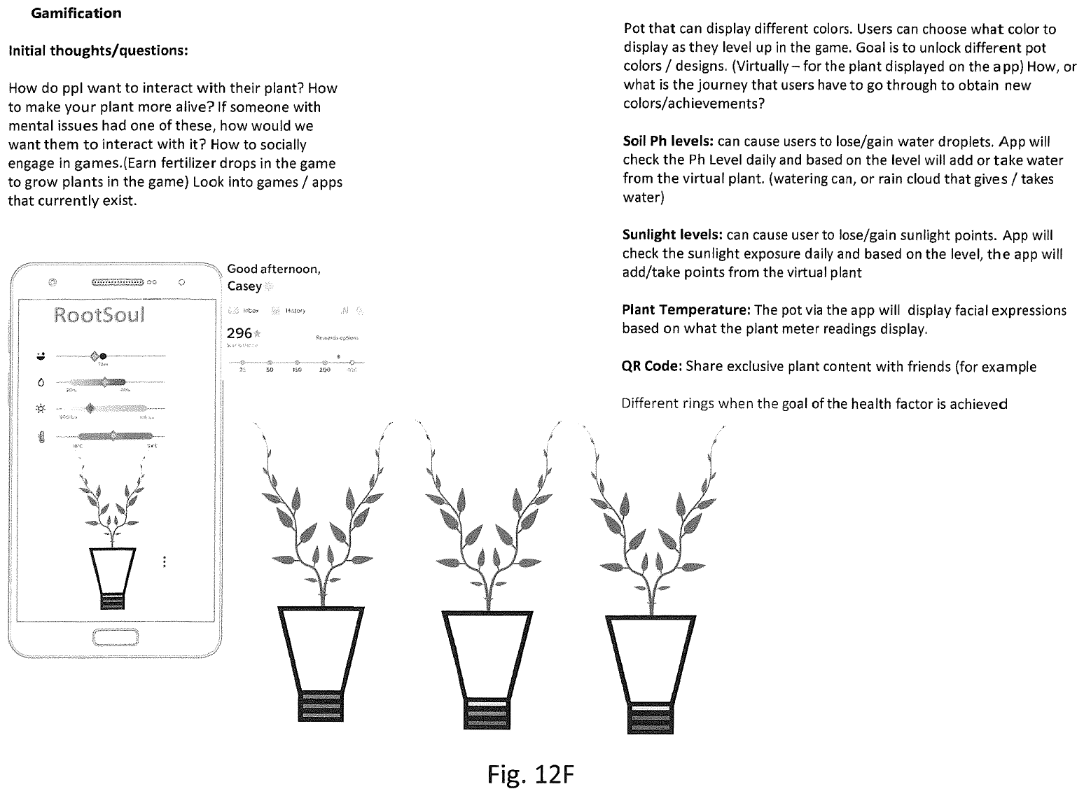

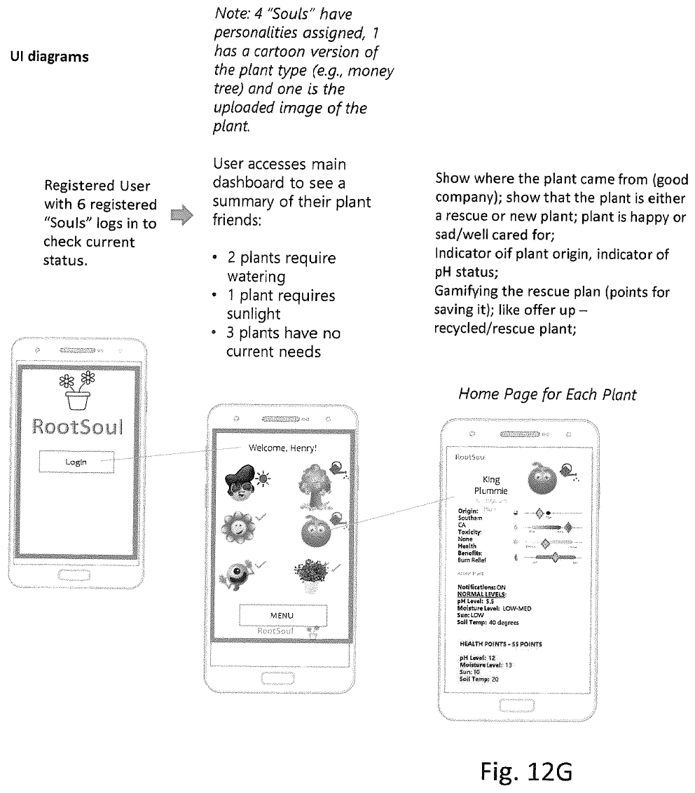

FIGS. 12A-12G show a system to track, manage, monitor, assess, etc., plant health, garden health, etc., which is implemented alone or in combination with any of the disclosed systems. In one embodiment, wireless transfer of sensory data are leveraged to measure, assess, display, etc., plant health, especially sunlight, humidity, temperature, fertilizer, etc. Referring to FIG. 12C, in an embodiment, system to track, manage, monitor, assess, etc., plant health, garden health, etc., includes an onboarding component including electrical circuitry configured to enable a mobile application (app) onboarding process. In an embodiment, the onboarding component includes a graphical user interface including user selectable icons representing onboarding setup inputs.

In a general sense, the various systems, devices, methods, technologies, methodologies, and the like described herein can be implemented by diverse types of electrical circuitry having a wide range of components, hardware, firmware, software, drivers, utilities, electrical components, electro-optical components, electro-mechanical components, and combination thereof, limited to patentable subject matter under 35 U.S.C. § 101.

Examples of electrical circuitry (e.g., computational circuitry, processing circuitry, control circuitry, and the like.) include application specific integrated circuits, discrete electrical circuits, integrated circuits, and the like.

In an embodiment, electrical circuitry includes, among other things, one or more computing devices such as a processor (e.g., a microprocessor), a central processing unit (CPU), a digital signal processor (DSP), an application-specific integrated circuit (ASIC), a field programmable gate array (FPGA), or the like, or any combinations thereof, and can include discrete digital or analog circuit elements or electronics, or combinations thereof. In an embodiment, electrical circuitry includes one or more ASICs having a plurality of predefined logic components. In an embodiment, electrical circuitry includes one or more FPGA having a plurality of programmable logic components.

In an embodiment, electrical circuitry includes one or more electrical components operably coupled (e.g., communicatively, electromagnetically, magnetically, ultrasonically, optically inductively, electrically, capacitively coupled, and the like) to each other. In an embodiment, electrical circuitry includes one or more remotely located components. In an embodiment, remotely located components are operably coupled via wireless communication. In an embodiment, remotely located components are operably coupled via one or more receivers, transceivers, or transmitters, antennas, or the like.

In an embodiment, electrical circuitry includes one or more network elements. Non-limiting examples of network elements include Local Area Networks (LANs), network gateway systems, network usage servers, Wide Area Networks (WANs), wireless base stations, wireless relays, and the like. In an embodiment, electrical circuitry includes computer and communication platforms that include data Input/Output (I/O) transceivers, digital processing circuitry, data storage memories, various software components, and the like.

In an embodiment, electrical circuitry includes one or more memory devices that, for example, store instructions or data for example, in an embodiment, the system 100 includes one or more memory devices storing user-specific sustainability information, user-specific carbon footprint information, enterprise-wide sustainability information, or enterprise-wide carbon footprint information with a remote client device and remote server. Non-limiting examples of one or more memory devices include volatile memory (e.g., Random Access Memory (RAM), Dynamic Random-Access Memory (DRAM), or the like); non-volatile memory (e.g., Read-Only Memory (RUM), Electrically Erasable Programmable Read-Only Memory (EEPROM), Flash memory, or the like); persistent memory; or the like. The one or more memory device can be coupled to, for example, one or more computing devices by one or more instructions, data, or power buses.

In an embodiment, electrical circuitry includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a touch-sensitive display, a mouse, a switch, a dial, or the like, and any other peripheral device. In an embodiment, electrical circuitry includes one or more user input/output components that are operably coupled to at least one computing device to control (electrical, electromechanical, software-implemented, firmware-implemented, or other control, or combinations thereof) at least one parameter associated with, for example, generating a user interface presenting a rating menu and receive one or more inputs indicative of a rating associated with the event based on the rating menu.

In an embodiment, electrical circuitry includes a computer-readable media drive or memory slot that is configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as a magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., receiver, transceiver, or transmitter, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

In an embodiment, electrical circuitry includes computing circuitry, memory circuitry, electrical circuitry, electro-mechanical circuitry, control circuitry, transceiver circuitry, transmitter circuitry, receiver circuitry, and the like. For example, in an embodiment, the plant community component 102, the virtual community component 110, the artificial intelligence component 112, the serenity component 114, the plant community exchange component 116, and the like comprises one or more of computing device circuitry, memory circuitry, and at least one of transceiver circuitry, transmitter circuitry, and receiver circuitry.

The herein described subject matter sometimes illustrates different components contained within, or connected with, different other components. It is to be understood that such depicted architectures are merely examples, and that in fact, a multitude of other architectures can be implemented that achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include, but are not limited to, physically mateable, physically interacting components, wirelessly interactable, wirelessly interacting components, logically interacting, logically interactable components, etc.

In an embodiment, one or more components may be referred to herein as “configured to,” “configurable to,” “operable/operative to,” “adapted/adaptable,” “able to,” “conformable/conformed to,” etc. Such terms (e.g., “configured to”) can encompass active-state components, or inactive-state components, or standby-state components, unless context requires otherwise.

The foregoing detailed description has set forth various embodiments of the devices or processes via the use of block diagrams, flowcharts, or examples. Insofar as such block diagrams, flowcharts, or examples contain one or more functions or operations, it will be understood by the reader that each function or operation within such block diagrams, flowcharts, or examples can be implemented, individually or collectively, by a wide range of hardware, software, firmware in one or more machines or articles of manufacture, or virtually any combination thereof. Further, the use of “Start,” “End,” or “Stop” blocks in the block diagrams is not intended to indicate a limitation on the beginning or end of any functions in the diagram. Such flowcharts or diagrams may be incorporated into other flowcharts or diagrams where additional functions are performed before or after the functions shown in the diagrams of this application. In an embodiment, several portions of the subject matter described herein is implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the type of signal-bearing medium used to conduct the distribution. Non-limiting examples of a signal-bearing medium include the following: a recordable type medium such as a floppy disk, a hard disk drive, a Compact Disc (CD), a Digital Video Disk (DVD), a digital tape, a computer memory, etc.; and a transmission type medium such as a digital or an analog communication medium (e.g., a fiber optic cable, a waveguide, a wired communications link, a wireless communication link (e.g., transmitter, receiver, transmission logic, reception logic, etc.), etc.).

While aspects of the present subject matter described herein have been shown and described, it will be apparent to the reader that, based upon the teachings herein, changes and modifications can be made without departing from the subject matter described herein and its broader aspects and, therefore, the appended claims are to encompass within their scope all such changes and modifications as are within the true spirit and scope of the subject matter described herein. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Further, if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense of the convention (e.g., “a system having at least one of A, B, and C” would include but not be limited to systems that have A alone, B alone, C alone, A and B together, A and C together, B and C together, and/or A, B, and C together, etc.). In those instances, where a convention analogous to “at least one of A, B, or C, etc.” is used, in general such a construction is intended in the sense of the convention (e.g., “a system having at least one of A, B, or C” would include but not be limited to systems that have A alone, B alone, C alone, A and B together, A and C together, B and C together, and/or A, B, and C together, etc.). Typically, a disjunctive word or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, the operations recited therein generally may be performed in any order. Also, although various operational flows are presented in a sequence(s), the various operations may be performed in orders other than those that are illustrated or may be performed concurrently. Examples of such alternate orderings includes overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are not intended to exclude such variants, unless context dictates otherwise.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

What is claimed is:

1. A system, comprising:
a plant community component including electrical circuitry configured to generate one or more garden attributes and one or more garden community recommendations based on at least one input indicative of a target garden characteristic and at least one input indicative of a calmness state.

2. The system of claim 1, further comprising:
a virtual community component including electrical circuitry configured to generate a virtual community environment on one or more client devices and to enable communication and digital content exchanges between multiple users in different physical locations.

3. The system of claim 1, wherein the plant community component is configured to generate the one or more garden attributes and the one or more garden community recommendations responsive to the least one input indicative of the target garden characteristic and at least one biometric input indicative of an anxiety state, a stress state, or an alertness state.

4. The system of claim 1, further comprising:
a serenity component including electrical circuitry configured to predict a calmness state;
wherein the plant community component includes electrical circuitry configured to generate one or more garden attributes and one or more garden community recommendations based on at least one input indicative of a target garden characteristic and at least one input indicative of the predicted calmness state; and
wherein the serenity component is configured to predict the calmness state based on at least one input indicative of an anxiety state, a stress state, an agitation level, or an excitement level.

5. The system of claim 1, further comprising:
a plant community exchange component including electrical circuitry configured to broadcast a plant community user request.

6. The system of claim 5, wherein the plant community exchange component includes electrical circuitry configured to broadcast an offer to rescue a plant or have a plant rescued, an offer to adopt a plant or to place a plant for adoption, or an offer to sell a plant or to buy a plant.

7. An interactive plant avatar system, comprising:
a plant personality unit including electrical circuitry configured to generate digital content consistent with a personality attribute set; and
a plant avatar unit including electrical circuitry configured to generate an interactive plant avatar on one or more client devices, the interactive plant avatar having one or more physical attributes inferred from the personality attribute set.

8. The interactive plant avatar system of claim 7,
wherein the plant personality unit includes electrical circuitry configured to generate digital content consistent with a physical attribute set; and
wherein the interactive plant avatar unit includes electrical circuitry configured to generate an interactive plant avatar on one or more client devices, the interactive plant avatar having one or more personality attributes inferred from the personality attribute set and one or more physical attributes inferred from the physical attribute set.

9. The interactive plant avatar system of claim 7, wherein the plant avatar unit is configured to change at least one visual feature of the interactive plant avatar based on an age-progression.

10. The interactive plant avatar system of claim 7, wherein the plant avatar unit is configured to vary an age indicator of the interactive plant avatar based on a digital age-progression process.

11. The interactive plant avatar system of claim 7, wherein the plant avatar unit is configured to change at least one audio feature of the interactive plant avatar based on a time-evolving program.

12. The interactive plant avatar system of claim 7, wherein the plant avatar unit is configured to change an audio response feature of the interactive plant avatar based on a time-evolving plant aging protocol.

13. The interactive plant avatar system of claim 7, wherein the plant avatar unit is configured to change an inflection, pitch, resonance, rhythm, tempo, texture, timber, tone, vocal fry, voice tremor, or volume of a plant audio response based a time-evolving plant aging protocol.

14. A wellness metaverse system, comprising:
a metaverse server coupled to a client computer;
a garden metaverse configuration component configured to generate a metaverse including one or more virtual plants having a geometric shape based on one or more inputs indicative of a wellness attribute;
wherein the metaverse is accessible by avatars of users that access the metaverse via client devices.

15. The wellness metaverse system of claim 14, wherein the wellness attribute comprises an anxiety mitigation space, a calming pace, a contemplation space, an energizing space, a fatigue mitigation space, a filtered air space, a health improvement space, a meditation space, a pollution abatement space, a relaxation space, or a stress mitigation space based on the at least one determined plant wellness potential, and at least one space wellness attribute.

16. The wellness metaverse system of claim 14, wherein the garden metaverse configuration component is configured to generate a metaverse including one or more virtual plants having a plant morphology including a leaf size, a leaf shape, a leaf color, a leaf size percent distribution, a leaf shape percent distribution, or leaf shape color percent distribution, or combinations thereof based on the one or more inputs indicative of the wellness attribute.

17. The wellness metaverse system of claim 14, wherein the garden metaverse configuration component is configured to generate a metaverse including one or more virtual plants having a plant morphology including a leaf size, a leaf shape, a leaf color, a leaf size percent distribution, a leaf shape percent distribution, or leaf shape color percent distribution, or combinations thereof based on a plant morphology plant wellness potential correlation policy.

18. The wellness metaverse system of claim 14, wherein the garden metaverse configuration component is further configured to generate one or more objects within the garden metaverse having a geometric shape that mimic a plant attribute associated with the wellness attribute.

19. The wellness metaverse system of claim 14, wherein the garden metaverse configuration component is configured to generate a metaverse including one or more virtual plants having a plant morphology including a leaf size, a leaf shape, a leaf color, a leaf size percent distribution, a leaf shape percent distribution, or leaf shape color percent distribution, or combinations thereof based on at least one input indicative of a target garden characteristic and at least one input indicative of the predicted calmness state.

20. The metaverse system of claim 14, further comprising:
electrical circuitry including a hardware processor, a display, and a graphical user interface (GUI) configured to render and access the wellness metaverse;
wherein the GUI includes a menu of selectable room attributes rendered on the display of a room within the wellness metaverse; and
wherein a user operates the GUI to navigate to a metaverse virtual space.
